(19) United States
(12) Patent Application Publication
(10) Pub. No.: US 2005/0118566 A1
(43) Pub. Date: Jun. 2, 2005

(54) Replicons derived from positive strand rna virus genomes useful for the production of heterologous proteins

(76) Inventors: Nicolas Escriou, Paris (FR); Sylvie Van Der Werf, Gif-sur-Yvette (FR); Marco Vignuzzi, Ontario (CA); Sylvie Gerbaud, Saint-Maur-des-Fosses (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 10/478,434

(22) PCT Filed: May 23, 2002
(86) PCT No.: PCT/IB02/02810

Related U.S. Application Data
(60) Provisional application No. 60/292,515, filed on May 23, 2001.

(51) Int. Cl. C12Q 1/70; C12Q 1/68; C12N 7/00; C07H 21/02

(52) U.S. Cl. 435/5; 435/6; 536/23.72; 435/235.1

(57) ABSTRACT

The present invention relates to replicons or self-replicating RNA molecules, derived from the genome of cardioviruses and aphtoviruses, which can be used to express heterologous proteins in animal cells. When injected in an animal host, for example in the form of naked RNA, these replicons permit the translation of the encoded heterologous protein. If the encoded heterologous protein is a foreign antigen, these replicons induce an immune response against the encoded heterologous protein. The invention uses cardiovirus and aphtovirus genomes to construct these replicons. The invention demonstrates that these replicons, when injected as naked RNA, can induce immune responses against a replicon-encoded heterologous protein in an animal recipient without the help of any kind of carrier or adjuvant.

The present invention relates to replicons or self-replicating RNA molecules, derived from the genome of cardioviruses and aphtoviruses, which can be used to express heterologous proteins in animal cells. When injected in an animal host, for example in the form of naked RNA, these replicons permit the translation of the encoded heterologous protein. If the encoded heterologous protein is a foreign antigen, these replicons induce an immune response against the encoded heterologous protein. The invention uses cardiovirus and aphtovirus genomes to construct these replicons. The invention demonstrates that these replicons, when injected as naked RNA, can induce immune responses against a replicon-encoded heterologous protein in an animal recipient without the help of any kind of carrier or adjuvant.

Genetic immunization is a powerful alternative tool for vaccine development It is based on the inoculation of DNA expression vectors containing gene sequences encoding the foreign protein. For instance, immunization with naked DNA vectors encoding the influenza nucleoprotein (NP) has been shown to induce antibodies and cellular responses, thereby protecting an animal host against both homologous and cross-strain challenge infection by influenza A virus variants (2, 27, 28). The advantages of DNA immunization include case of production, ease of purification and administration of the vaccine, and the resulting long-lasting immunity.

The long-term immunity associated with DNA immunizations is likely related to the long-term persistence and expression of injected DNA. Indeed, injected DNA molecules have been shown to persist more than one year in the mouse model (31). However, for this very reason some question remains, from a clinical standpoint, as to the potential risk of integration of DNA sequences into the host genome. Although preliminary studies in animals have not demonstrated genome integration events (19), such integrations can cause insertional mutagenesis, activation of protooncogenes, or chromosomal instability, which may result in diseases, such as cancer (35).

To avoid this potential problem, the inventors generated naked, self-replicating RNA molecules, or replicons, derived from positive strand RNA virus genomes. RNA has already been proposed as an alternative to DNA for genetic immunization, but development of this approach has faced new problems posed by the short intracellular half-life of RNA and its degradation by ubiquitous RNases. Initial attempts used mRNA to induce immune responses, administered intramuscularly (5), by gold particle-coated gene gun delivery (25) or by liposome-encapsulated injection to protect the RNA during administration (17). To further improve delivery of these molecules and expression of the encoded heterologous proteins, encapsidated self-replicating RNAs or replicons derived from the genomes of positive strand RNA viruses have been developed to vehicle heterologous sequences into the cell. In these replicons, genomic structural genes are replaced by heterologous sequences, while retaining their non-structural genes to permit one round of replication. This molecular design permits the expression of foreign proteins.

The genomes of the alphaviruses, Semliki Forest virus (SFV), Sindbis virus and Venezuelan equine encephalitis virus, have been manipulated in this manner to allow the expression of foreign proteins (11, 24). Protein packaging of RNA-based replicons stabilizes them, allowing the injection of the resulting virus-like particles to induce an array of immune responses against the heterologous protein. Similarly, the positive sense RNA of poliovirus has been deleted of its capsid coding sequences to permit the expression of foreign proteins (3, 21) and when packaged into virus-like particles, can induce immune responses upon injection of mice transgenic for the poliovirus receptor (18, 23).

Contrary to studies with packaged RNA molecules, the inventors have studied the ability of naked RNA replicons to induce immune responses, arguing that packaging these vectors is unnecessary since their replicative nature alleviates the need for large quantities of input RNA. In the case of recombinant SFV vectors encoding the hemagglutinin (HA) and NP molecules of influenza A virus, naked RNA injection has been found to induce specific antibodies (6, 34). Recently, some publishers have reported that recombinant replicons derived from SFV were able to induce protective antibodies against Influenza A, Respiratory Syncytial and Looping III viruses (10), and cytotoxic T lymphocytes (CTLs) against lacZ used as model antigen (33).

The inventors reported recently (30) that a recombinant SFV replicon, which encodes the internal influenza A NP protein (rSFV-NP), could elicit both humoral and cellular immune responses against Influenza A virus upon injection of RNA in naked form, in a response that was found to be comparable to that induced by plasmid DNA. Furthermore, the inventors demonstrated that naked injection of the rSFV-NP replicon was able to induce a CTL response specific of the immunodominant epitope of the influenza NP and to reduce pulmonary viral loads in mice challenged with a mouse-adapted influenza virus, to the same extent as does the better described DNA immunization technique.

The inventors reported also that a poliovirus replicon, which encodes the internal influenza A NP protein (rΔP1-E-NP), could elicit a much weaker humoral immune response in mice than did the Semliki rSFV-NP replicon upon injection of RNA in naked form. Moreover, no CTL response against the Influenza NP could be detected in mice injected with rΔP1-E-NP replicon RNA (30). Therefore, the inventors decided to explore the use of the genome of other virus members of the Picornaviridae family in order to construct new replicons for the expression of heterologous proteins in animal cells and in animal recipients, after their injection, in the form of naked RNA, for example. Members of the Aphtovirus and Cardiovirus genus, which share the same genetic organization could be used for this purpose. As a working example, the inventors used the Mengo virus as the prototype cardiovirus.

To construct a replicon based on the Mengo virus genome, the inventors determined which genomic sequences could be deleted without affecting the molecule's replication. To this end, a series of in frame deletions encompassing all or part of the coding region of the L-P1-2A precursor protein were engineered in the Mengo virus genome. The replicative characteristics of the corresponding subgenomic RNA molecules were analyzed. The inventors demonstrated that all the coding region of the L-P1-2A precursor could be removed from the Mengo virus genome without affecting its replicative capacity, with the exception of a short nucleotide sequence of the VP2 coding region. Indeed, the inventors demonstrated that the region encompassing nucleotides 1137 to 1267 of the Mengo virus genome (numbering is for the vMC24 attenuated strain) contained a Cis-acting Replication Element (CRE), which was absolutely required for a subgenomic Mengo virus RNA molecule to be able to replicate in transfected cells (15). The situation here is strikingly different from what was observed with the poliovirus genome and the aphtovirus genome, for which the entirety of the capsid protein precursor (P1) could be deleted without affecting the replication of the corresponding subgenomic RNA molecules (1, 12).

After constructing the Mengo virus-derived replicon, the inventors demonstrated that subgenomic Mengo virus replicons were able to express heterologous sequences. The immunogenicity of replicons can be improved by various methods. For example, the inventors have demonstrated that Mengo virus replicons can be encapsidated in trans when transfected into cells expressing the P1 precursor of capsid proteins. Replicon RNAs can also be condensed with polycationic peptide protamine as described by Hoerr et al. (37).

The invention describes the construction and the use of replicons constructed from genomes of viruses in the genus Cardiovirus. Similar replicons can also be constructed from viral genomes in the genus Aphtovirus, as aphtoviruses are also members of the Picornaviridae family and share identical genetic organization with cardioviruses.

The term “replicons” as used herein includes, but is not limited to, self-replicating recombinant positive strand RNA molecules.

The term “positive strand” as used herein includes, but is not limited to an RNA stand that directly encodes a protein.

The term “express” or any variation thereof as used herein includes, but is not limited to, giving rise to or encoding the production of a protein or part of a protein.

The present invention provides a self-replicating recombinant positive strand RNA molecule of a viral genome of an RNA virus (replicon), wherein the RNA molecule comprises:

(a) RNA sequence encoding the non-structural proteins of the RNA virus;

(b) viral non-encoding RNA sequences necessary for viral replication; and

(c) RNA sequence encoding a heterologous protein or fragment of a heterologous protein.

According to an advantageous embodiment of said replicon, the RNA sequence encoding the non-structural proteins in a) and/or the viral non-encoding RNA sequences necessary for viral replication in b) are either in mutated or truncated forms.

According to an other advantageous embodiment of said replicon, the RNA virus is in the genus of Cardiovirus or Aphtovirus; preferably a Mengo virus; most preferably, said replicon further comprises the Cis-acting Replication Element (CRE) of the Mengo virus or the Theiler's virus VP2 gene.

According to an other advantageous embodiment of said replicon, the heterologous protein as defined in c), is chosen from a biologically active protein, a reporter protein, a cytotoxic protein, a protein of a pathogen, or a protein of a tumor; preferably the reporter protein is green fluorescent protein and the protein of a pathogen is influenza nucleoprotein or influenza hemagglutinin.

According to an other advantageous embodiment of said replicon, the fragment of a heterologous protein as defined in c), is an antigen or epitope of said heterologous protein.

Replicons can be constructed by deleting all or part of capsid coding sequences and retaining all coding and non-coding sequences necessary for replication. Retention of genomic replication allows the expression of viral and heterologous gene products in appropriate cells. For example, the CRE, found in the Mengo virus VP2 gene, is essential for replication.

Replicons can be prepared by several methods. In one embodiment, the appropriate DNA sequences are transcribed in vitro using a DNA-dependant RNA polymerase, such as bacteriophage T7, T3, or SP6 polymerase. In another embodiment, replicons can be produced by transfecting animal cells with a plasmid containing appropriate DNA sequences and then isolating replicon RNA from the transfected cells. For example, the complementary DNA (cDNA) encoding a replicon can be placed under the transcriptional control, downstream, of the polymerase I promoter and upstream of the cDNA of the hepatitis δ ribozyme. The term “transfection” as used herein includes, but is not limited to, the introduction of DNA or RNA into a cell by means of electroporation, DEAE-Dextran treatment, calcium phosphate precipitation, liposomes (e.g., lipofectin), protein packaging (e.g., in pseudo-viral particles), protamine condensation, or any other means of introducing DNA or RNA into a cell.

The invention also provides the following DNA molecules which are useful for the production of the self-replicating recombinant positive strand RNA molecule according to the invention:

a DNA molecule that encodes a self-replicating recombinant positive strand RNA molecule of a viral genome of an RNA virus according to the invention. In a preferred embodiment, said DNA molecule further comprises a suitable cloning vector,

a DNA molecule comprising the sequence selected from SEQ. ID. NO. 26 and SEQ ID NO: 27 (plasmids deposited at the CNCM Institut Pasteur, 28, rue du Docteur Roux, 75724 Paris cedex 15, France, on May 21, 2001, respectively under Accession No. I-2668 and 2669) or a fragment thereof, and DNA sequence encoding a heterologous protein or fragment of a heterologous protein in an expressible form; preferably said DNA molecule comprises SEQ ID NO: 28 (plasmid deposited at the CNCM Institut Pasteur, 28, rue du Docteur Roux, 75724 Paris cedex 15, France, on May 16, 2002, under Accession No. 1-2879), and
a DNA molecule comprising the sequence selected from the sequence SEQ. ID. NO. 26 and SEQ ID NO: 27 (plasmids deposited at the CNCM Institut Pasteur, 28, rue du Docteur Roux, 75724 Paris cedex 15, France, on May 21, 2001, respectively under Accession No. I-2668 and 2669) either in a mutated or truncated form, or a fragment thereof, and DNA sequence encoding a heterologous protein or fragment of a heterologous protein in an expressible form.

According to preferred embodiments of said DNA molecules, the heterologous protein is chosen from a biologically active protein, a reporter protein, a cytotoxic protein, a protein of a pathogen, or a protein of a tumor; preferably, the reporter protein is green fluorescent protein, the protein of a pathogen is influenza nucleoprotein, influenza hemagglutinin, or lymphocitic choriomeningitis virus nucleoprotein and the heterologous protein fragment is an antigen or epitope of said heterologous protein, preferably the NP118-126 epitope of the lymphocytic choriomeningitis virus nucleoprotein.

The replicon of the invention has several potential uses. In a first embodiment, replicons can be used to express heterologous proteins in animal cells or an animal host by inserting sequences coding for heterologous polypeptides into the replicons and introducing the replicons into the animal cells or the animal host. In one embodiment, the animal host is a dog, cat, pig, cow, chicken, mouse, or horse. In a preferred embodiment, the animal host is a human. Replicons can be introduced into the host by several means, including intramuscular injection, gold particle-coated gene gun delivery, protein-packaged injection (e.g., packaged in pseudo-viral particles), protamine-condensed injection, or liposome-encapsulated injection. For example, a Mengo virus-derived replicon allows the transient expression of a therapeutic protein at or near to the site of injection or expression of a toxic protein or a proapoptotic protein in a solid tumor by direct injection, thus providing a form of anti-tumor gene therapy. In addition, recombinant replicons can be used in vitro or in vivo in order to express conveniently detected reporter protein. These replicons cm be used to monitor RNA replication and RNA delivery, thereby allowing for optimization of animal cell transfection or RNA delivery into an animal host. Finally, replicons can be used to express any protein of interest for further studies on protein characterization, protein production, or protein localization, for example.

In another embodiment replicons can be used to induce an immune response against the encoded heterologous protein in an animal recipient Thus, the replicons of the instant invention along with a pharmaceutically acceptable carrier can constitute a vaccine. Pharmaceutical carriers include, but are not limited to, sterile liquids, such as water, oils, including petroleum oil, animal oil, vegetable oil, peanut oil, soybean oil, mineral oil, sesame oil, saline solutions, aqueous dextrose, glycerol solutions, polycationic particles, protein particles, protamine particles, liposomes, gold particles, or any other protein or molecule able to condense the RNA. Replicons can, for example, be injected in the form of either “naked” or encapsidated RNA. The term “naked” as used herein includes, but is not limited to, an RNA molecule not associated with any proteins.

In one example, a replicon can express antigenic determinants of any pathogen, including bacteria, fungi, viruses, or parasites. Replicons can also express tumor antigens or a combination of tumor antigens and pathogen antigens. Such a replicon can induce an immune response against a pathogen or tumor, thereby comprising a vaccine against the corresponding disease. In this regard, the ability of Mengo virus-derived replicons to induce a strong cellular immune response is an advantageous property.

In a second example, a replicon can also be used as an immunotherapeutic agent to treat individuals who are already ill. Specifically, replicons can strengthen an existing immune response or induce a new response against a pathogen or tumor antigen already present in the individual, thereby comprising a therapy against the corresponding disease. For example, hepatitis B can be treated in this manner by administering a replicons that express the hepatitis B virus surface antigen.

In a third example, a replicon can be constructed in order to express a synthetic polypeptide consisting of a string of T cell epitopes derived from the same antigen or from different antigens. These epitopes can specifically stimulate CD4+ T cells (helper T cells) or CD8+ T cells (CTLs). Such a replicon can (1) induce a multispecific immune response while taking into account HLA variability and (2) limit the pathogen's or tumor cell's evasion of the immune response via antigenic escape.

In a fourth example, any biologically active protein can be expressed by a replicon. In one embodiment the biologically active protein is an immunomodulatory protein, such as a cytokine or a chemokine, which can modulate the immune response of the host. If injected at the same time and location as a replicon expressing a foreign antigen, the cytokine replicon can modulate the immune response induced against the foreign antigen. These replicons can also be used alone to modulate the immune response against any pathogen antigen or cancer antigen. These replicons can also modulate autoimmune pathology, if properly administered.

Thus, the invention provides a vaccine comprising at least one self-replicating recombinant positive strand RNA molecule according to the invention, and a pharmaceutically acceptable carrier.

In an advantageous embodiment of said vaccine, the self-replicating recombinant positive strand RNA molecule is naked RNA.

In an other advantageous embodiment of said vaccine, the self-replicating recombinant positive strand RNA molecule is encapsidated.

The invention also provides a method of inducing a protective immune response in a host comprising:

(a) preparing at least one molecule selected from the self-replicating recombinant positive strand RNA molecule and the DNA molecule according to the invention, in a pharmaceutically acceptable carrier, and

(b) immunizing the host with the preparation of step (a).

In an advantageous embodiment of said method, the self-replicating recombinant positive strand RNA molecule and the DNA molecule of step a) are naked.

In an other advantageous embodiment of said method, the self-replicating recombinant positive strand RNA molecule of step a) is encapsidated.

The invention also provides a therapeutic composition comprising at least one molecule selected from the self-replicating recombinant positive strand RNA molecule and the DNA molecule according to the invention, in an acceptable medium.

The invention also provides a therapeutic kit comprising at least one molecule selected from the self-replicating recombinant positive strand RNA molecule and the DNA molecule according to the invention in an acceptable medium.

The invention also provides a method for modulating the immune response in a host comprising:

(a) preparing at least one one molecule selected from the self-replicating recombinant positive strand RNA molecule and the DNA molecule according to the invention in a pharmaceutically acceptable carrier; and

(b) immunizing the host with the preparation of step (a).

In an other advantageous embodiment of said methods, the pharmaceutically acceptable carrier is chosen from water, petroleum oil, animal oil, vegetable oil, peanut oil, soybean oil, mineral oil, sesame oil, saline solutions, aqueous dextrose, glycerol solutions, polycationic particles, protein particles, protamine particles, liposomes, and gold particles.

In an other advantageous embodiment of said methods the host is selected from a human, a pig, a dog, a cat, a cow, a chicken, a mouse, or a horse.

The invention also provides a method for improving the immunogenicity of a self-replicating recombinant positive strand RNA molecule of a viral genome of an RNA virus by producing an encapsidated self-replicating recombinant positive strand RNA molecule of a viral genome of an RNA virus comprising:

(a) transfecting the DNA or the self-replicating recombinant positive strand RNA molecule according to the invention into cells expressing the P1 precursor of capsid proteins;

(b) preparing the encapsidated self-replicating recombinant positive strand RNA molecule from the transfected cells; and

(c) immunizing a host with the preparation of step (b).

The invention also provides a method for improving the immunogenicity of a self-replicating recombinant positive strand RNA molecule of a viral genome of an

(a) condensing the self-replicating recombinant positive strand RNA molecule according to the invention; and

(b) immunizing a host with the condensed RNA molecule of step (a).

The invention is further demonstrated by way of drawings and working examples in which replicons were engineered from the Mengo virus genome. It should be understood however that these examples are given only by way of illustration of the invention and do not constitute in anyway a limitation thereof.

### BRIEF DESCRIPTION OF DRAWINGS

FIG. 1 is a schematic representation of plasmids encoding subgenomic recombinant replicons derived from the Mengo virus genome. Green fluorescent protein (GFP), HA, and NP genes are shown as hatched boxes. The CRE is shown as a stippled box. The HA protein signal peptide (SP) and HA transmembrane region (TM) are indicated by black bands.

FIG. 2 is an SDS-PAGE analysis demonstrating the in vitro translation and processing of the recombinant Mengo virus polyproteins in rabbit reticulocyte lysates. Positions of molecular mass markers are indicated on the right. Mengo virus protein precursors as well as some of their major cleavage products are indicated on the left. The GFP-NP and GFP polypeptides and the influenza NP encoded by the recombinant replicons are indicated by solid arrows.

FIG. 3 is a slot blot demonstrating the replication of subgenomic Mengo virus-derived replicons. At the indicated times post-transfection, cytoplasmic RNA was harvested for analysis.

FIG. 4 is a fluorocytometer reading of GFP expression in HeLa cells transfected with recombinant replicon rMΔBB, rMΔBB-GFP or rMΔXBB-GFP.

FIG. 5 is an SDS-PAGE analysis of an immunoprecipitated influenza NP protein expressed in [³⁵] methionine labeled HeLa cells transfected with recombinant replicon rMΔBB-NP. Loaded samples are as follows: mock transfected HeLa cells (lane 1); HeLa cells transfected with replicons rMΔBB (lane 2). rMΔBB-NP (lane 3) or rMΔBB-GFP-NP (lane 4) and harvested at 10 hours post-transfection; mock infected HeLa cells (lane 5) and HeLa cells infected with A/PR/8/34 virus (lane 6) and harvested at 20 hours post-infection. Molecular masses and positions of the viral HA protein, the viral NP protein, and the viral M1 protein are shown on the right.

FIG. 6 is a CTL assay demonstrating the induction of NP-specific CTL activity in C57BL/6 mice immunized with rMΔBB-NP. Groups of four C57BL/6 mice were immunized at three week intervals with the following vaccination protocols: 1 injection of 50 μg of pCI (◯) or pCI-NP (●) DNA; 2 injections of 25 μg of rMΔBB (□) or rMΔBB-NP (▪) RNA. Splenocytes were harvested three weeks after the last injection, stimulated in vitro and then tested for cytolytic activity in a chromium release assay against syngenic EL4 target cells loaded with NP366 peptide (a) or not (b). The percentage of specific lysis is shown at various effector: target ratios. Data shown is from one out of two experiments. Three weeks after the last injection, the frequency of influenza virus-specific CDS+ T cells was measured by the IFNγ ELISPOT assay in the presence of the immunodominant NP366 peptide (c), as described in Materials and Methods. Data are expressed as the number of SFC per 10⁵ spleen cells.

FIG. 7 is an ELISA demonstrating the induction of NP-specific antibodies in C57BL/6 mice immunized with rMΔBB-NP, according to the same vaccination protocol as in FIG. 6. Titers are represented as the reciprocal of the highest dilution of pooled serum, for a given group of five or six mice, giving an optical density value at 450 nm equal to two times that of background levels in a direct ELISA test using purified split A/PR/8/34 virions as antigen.

FIG. 8 is a graphical representation of the pulmonary viral loads in mice immunized with rMBBΔ-NP and then challenged with influenza virus. Open circles represent mean values of each group, bars indicate standard deviations. Data shown is from one out of two experiments.

FIG. 9A is an SDS-PAGE analysis demonstrating the in vitro translation of the native form of HA in rabbit reticulocyte lysates. The influenza HA polypeptide encoded by the rMΔFM-HA recombinant replicon is indicated by a solid arrow and a non-cleaved precursor by an open arrow.

FIG. 9B is a slot blot demonstrating that monocistronic Mengo virus replicons cannot express foreign glycosylated protein in transfected eukaryotic cells. At the indicated times post-transfection, cytoplasmic RNA was harvested and slot blotted onto a nylon membrane for analysis.

FIG. 10 is an SDS-PAGE analysis of immunoprecipitated GFP fusion polypeptides expressed in [³⁵S] methionine labeled HeLa cells transfected with recombinant Mengo virus replicons. Loaded samples were as follows: mock-transfected HeLa cells or HeLa cells transfected with replicon RNAs rMΔBB-GFP, rMΔBB-GFP-NP118 (2 clones) or rMΔB-GFP-1cmvNP. Molecular masses (kDa) are shown on the left.

FIG. 11 is an ELISPOT assay demonstrating the induction of LCMV-specific T cells in BALB/c mice immunized with rMΔBB-GFP-NP118 and rMΔBB-GFP-1cmvNP replicon RNA and, as controls, with pCMV-NP and pCMV-MG34 plasmid DNA. Three weeks after the last injection, the frequency of LCMV-specific CD8+ T cells was measured by the IFNγ ELISPOT assay in the presence of the immunodominant NP118-126 peptide, as described in Materials and Methods. Data are expressed as the number of SFC per 10⁵ spleen cells.

FIG. 12 is a fluorocytometer reading of GFP expression in HeLa cells transfected with recombinant Mengo virus replicons rMΔBB-GFP, rMΔBB-GFP-NP118, or rMΔBB-GFP-1cmvNP.

### EXAMPLES

Replicon cDNA derived from the Mengo virus genome was cloned, in positive sense orientation, into a bacterial plasmid downstream of the T7 RNA polymerase I promoter and upstream of a unique BamH I cleavage site. After linearizing the bacterial plasmid with BamH I, T7 RNA polymerase was used to synthesize a viral RNA-like transcript, which can be used for transfection of animal cells or for injection into an animal host.

The first series of replicons, the rMΔBB series, were constructed as described in Materials and Methods and Example 1. Almost all the coding sequences of the L-P1-2A precursor were deleted with the exception of the CRE. These replicons did replicate in transfected HeLa cells and subsequently expressed GFP or influenza NP as fusion proteins with vector derived residues. The rMΔBB-NP replicon, when injected in the form of naked RNA, induced an anti-NP immune response in mice. Based on this strategy, other replicons were constructed; they did replicate and subsequently permitted the expression of the NP of lymphocytic choriomeningitis virus (LCMV) and of a synthetic polypeptide corresponding to the immunodominant NP118-126 epitope of LCMV for H2^{d} mice, as described in Example 9.

The second replicon series, the rMΔFM series, were constructed to express foreign sequences in a more native form by minimizing the amount of vector sequences fused to the foreign protein sequences. These rMΔFM replicons also replicated in transfected HeLa cells. In contrast, the rMΔFM-HA recombinant replicon, which contains the entirety of the influenza HA sequences including its SP and TM region, was not replication competent

Picornaviral genomes normally do not encode glycoproteins. The inventors noted that monocistronic Mengo virus-derived replicons cannot express foreign glycosylated proteins, as the inventors previously showed for replicons derived from the poliovirus genome. However, the inventors have previously demonstrated that dicistronic poliovirus (PV) replicons can express glycoproteins. Specifically, the inventors constructed a dicistronic replicon, rΔPV-IR-RA, for which translation of the HA and PV sequences were uncoupled by the insertion of the EMCV Internal Ribosome Entry Site (IRES). The rΔPV-IR-HA replicon replicates upon transfection and permits the expression of the HA, correctly glycosylated, at the cell surface (29). Likewise, dicistronic Mengo virus replicons can be constructed by the insertion of a foreign viral or mammalian IRES and tested for the ability to replicate and direct the session of glycosylated proteins, such as viral or tumor antigens or biologically active polypeptides.

Materials and Methods

Cells, Viruses and Plasmid HeLa cells (ATCC Accession No. CCL-2) were grown at 37° C. under 5% CO₂ in DMEM complete medium (Dulbecco's modified Eagle medium with 1 mM sodium pyruvate, 4.5 mg/ml L-glucose, 100 U/ml penicillin and 100 μg/ml streptomycin), supplemented with 5% heat-inactivated fetal calf serum (FCS) (TechGen #8010050). EL4 (mouse lymphoma, H-2^{b}) (ATCC Accession No. TIB-39) and P815 (mouse mastocytoma, H-2^{d}) (ATCC Accession No. TIB-64) cells were maintained in RPMI complete medium (RPMI 1640, 10 mM HEPES, 50 μM β-mercaptoethanol, 100 U/ml penicillin, 100 μg/ml streptomycin), supplemented with 10% FCS.

Mouse-adapted influenza virus A/PR/8/34(ma) (H1N1) was derived from serial passage of pulmonary homogenates of infected to naive mice as described previously (20). Subsequent viral stocks were produced by a single allantoic passage on 11 day-old embryonated hen's eggs, which did not affect its pathogenicity for mice. Plasmid pCI-NP was constructed by the insertion of the coding sequences of the influenza NP between the Sal I and Sma I sites of expression plasmid pCI (Promega #E1731) downstream of the CMV immediate-early enhancer/promoter, as described elsewhere (30). Plasmid pCI-NP contains the consensus sequence of A/PR/8/34(ma) NP cDNA, which can be obtained from the inventors upon request, with a silent mutation at codon 107 (E: GAG→GAA) and an additional Pro→Ser mutation at codon 277. The codon 277 mutation does not directly affect the major histocompatibility class I (MHC-I) restricted immunodominant epitope of interest, NP366-374.

Construction of Plasmids for the In Vitro Transcription of Recombinant Replicons

Plasmids containing Mengo virus cDNAs with L-P1-2A deletions and substitutions were derived from plasmid pMC24 (also named pM16.1; kindly provided by Ann Palmenberg, University of Wis., Madison, Wis.), which contains the full-length infectious cDNA of an attenuated Mengo virus strain placed downstream from the phage T7 promoter (8).

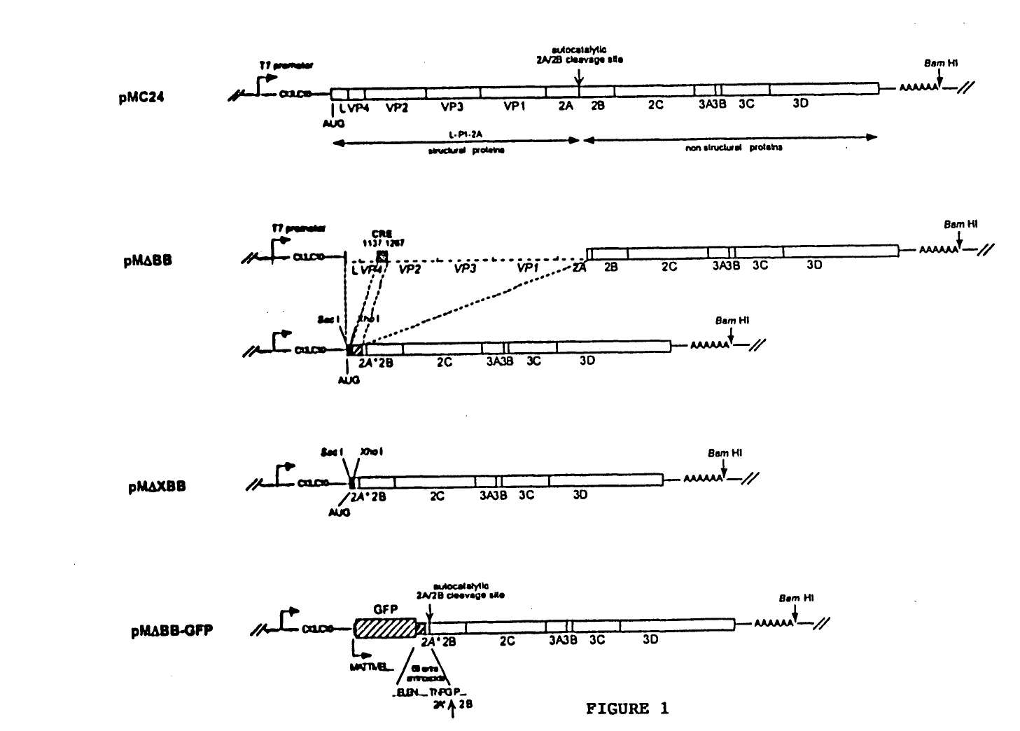

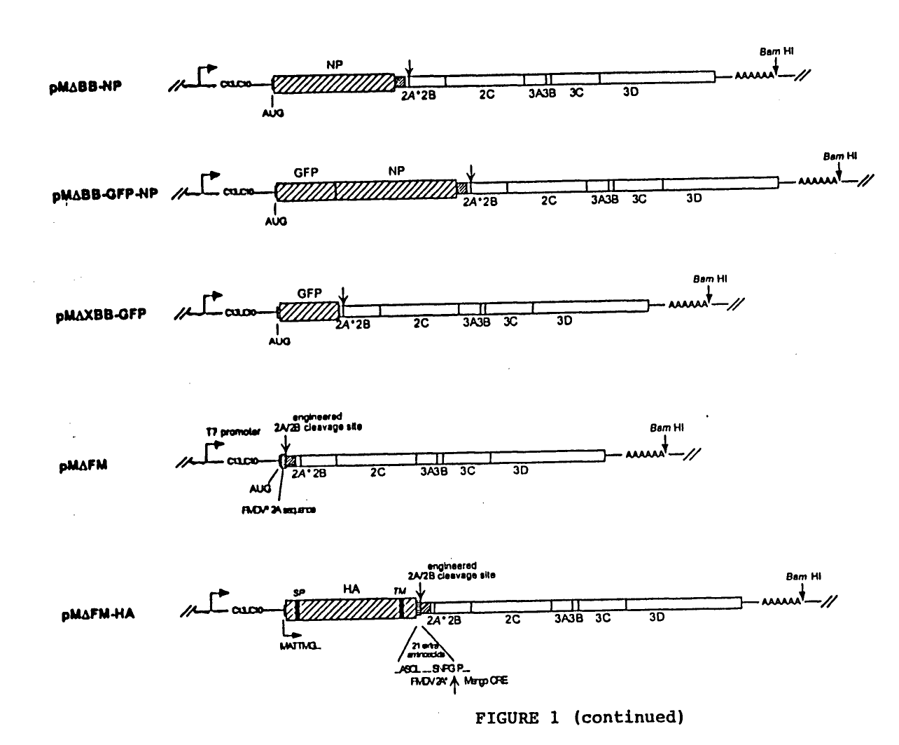

Plasmid pMΔBB (SEQ ID NO: 26) contains a subgenomic Mengo virus cDNA in which nucleotides 737 to 3787 were replaced by a Sac I/Xho I polylinker (GAGCTCGAG) (SEQ. ID. NO. 1) and nucleotides 1137-1267 of vMC24 cDNA encompassing the Mengo virus CRE (FIG. 1). Plasmid pMΔBB was constructed by digesting plasmid pMN34 (15) with BstB I followed by self-ligation. Bacteria containing the pMΔBB were deposited at the Collection Nationale de Cultures de Microorganismes (CNCM) Paris, France, on May 21, 2001, under Accession Number I-2668. Plasmid pMΔN34 is similar in design to pMΔBB, but a smaller portion of the Mengo virus genome (nucleotides 737 to 3680) has been removed.

Plasmid pMΔXBB was constructed so as to remove CRE encompassing sequences from the pMΔBB plasmid. Briefly, a Xho I-Bst BI linker was obtained by the annealing of the oligonucleotides 5′-TCGAGGCTAGCTT-3′ (SEQ. ID. NO. 2) and 5′-CGAAGCTAGCC-3′ (SEQ. ID. NO. 3) and cloned between the Xho I and Bst B I site of plasmid pMNΔ34. Positive clones were sequenced using a Big Dye terminator sequencing kit (Perkin Elmer #P/N 4303150) and an ABI377 automated sequencer (Perkin-Elmer).

For cloning purposes, the sequences encoding GFP were amplified by PCR with the proof-reading PWO polymerase (Roche #1644947) using plasmid pEGFP-N1 (Clontech #6085-1) as a template and oligonucleotides

5′-GCTGAGCTCATGGTGAGCAAGGGCGAGGAGC-3′ (SEQ. ID. NO. 4); and

5′-GCAGAGCTCCTTGTACAGCTCGTCCATGCCG-3′ (SEQ. ID. NO. 5), both of which included a Sac I restriction enzyme site (underlined), as primers. GFP sequences were inserted in frame at the Sac I site of plasmids pMΔBB and pMΔXBB, yielding respectively plasmid pMΔBB-GFP and pMΔXBB-GFP. Positive clones were sequenced as indicated above.

The PMΔBB-NP plasmid was constructed in two steps. First, a recombinant cDNA fragment containing a mutated cDNA of the influenza virus A/PR/8/34(ma) NP was generated with PWO polymerase following an overlap extension PCR protocol (22). The mutagenesis was performed in order to revert the mutation present at codon 277 to the correct Pro277 and to introduce a silent mutation at codon 160 (D: GAT→GAC), thus destroying a BamH I site for the purpose of the subsequent experiments. Briefly, the two overlapping DNA fragments were generated by PCR amplification of plasmid pCI-NP with oligonucleotides

5′-TCTCCACAGGTGTCCACTCC-3′ (SEQ. D. NO. 6) and

5′-CACATCCTGGGGTCCATTCCGGTGCGAAC-3′ (SEQ. ID. NO. 7), and plasmid pTG-NP24 (which is similar to pTG-NP82 described in reference 30, but does not contain the P277S mutation) with oligonucleotides

5′-ACCGGAATGGACCCCAGGATGTGCTCTCTG-3′ (SEQ. ID. NO. 8) and

5′-GTCCCATCGAGTGCGGCTAC-3′ (SEQ. ID. NO. 9). The fusion PCR product, generated with oligonucleotides

(SEQ. ID. NO. 10)

5′-CGGAATTCTCGAGATGGCGTCTCAAGGCACCAAACG-3′;
and

(SEQ. ID. NO. 11)

5′-GCGAATTCTCGAGATTGTCGTACTCCTCTGCATTGTC-3′

both of which included a Xho I restriction enzyme site (underlined), was cloned into the EcoR I site of plasmid pTG186 (13), yielding plasmid pTG-R4. Positive clones were sequenced as indicated above. Second, plasmid pMΔBB-NP was generated by inserting the sequences encoding NP, derived from pTG-R4 upon digestion with Xho I, into the Xho I site of pMΔBB such that the NP sequence was in frame with the remainder of the Mengo virus polyprotein sequence. The GFP coding sequences were inserted into the pMΔBB-NP plasmid in the same manner as for the pMΔBB plasmid using a unique Sac I site (see above). yielding plasmid pMΔBB-GFP-NP. For construction of the pMΔBB-GFP-1cmvNP plasmid, the coding sequences of the NP of the LCMV virus were amplified by PCR using the oligonucleotides
5′-CGGAATTCTCGAGATGTCCTTGTCTAAGGAAGTTAAG-3′ (SEQ. ID. NO 12) and
5′-GCGAATTCTCGAGTGTCACAACATTTGGGCCTC-3′ (SEQ. ID NO. 13) with plasmid pCMV-NP (39) as a template. The resulting DNA fragments were cloned into the Xho I site of plasmid pMΔBB-GFP. Positive clones were sequenced as indicated above.

To reconstitute the coding sequence of the NP118-126 H2⁴-restricted immunodominant epitope of LCMV, a synthetic linker was obtained by annealing the oligonucleotides

5′TCGAAGCTAGCGAAAGACCCCAAGCTTCAGGTGTGTATATGGGTAATTTGA CAC-3′ (SEQ. ID. NO. 14) and

5′TCGAGTGTCAAATTACCCATATACACACCTGAAGCTTGGGGTCTTTCGCTAG CT-3′ (SEQ. ID. NO. 15) at a 100 μM concentration in 750 mM Tris-HCl pH 7.7 for 5 minutes at 100° C. then for one hour at 20° C. This linker was inserted at the Xho I site of the pMΔBB-GFP plasmid, yielding plasmid pMΔBB-GFP-NP118. Positive clones were sequenced as indicated above.

For construction of the pMΔFM plasmid (SEQ ID NO: 27), a synthetic linker was obtained by annealing together the oligonucleotides

5′TCGAGGCTAGCCAGCTTTGAATTTTGACCTTCTTAAGCTTGCGGGAGACGTC GAGTCCAACCCTGGGCCCT-3′ (SEQ. ID. NO. 16) and

5′TCGAAGGGCCCAGGGTTGGACTCGACGTCTCCCGCAAGCTTAAGAAGGTCA A AATTCAACAGCTGGCTAGCC-3′ (SEQ. ID. NO. 17) at a 100 μM concentration in 750 mM Tris-HCl pH 7.7 for 5 minutes at 100° C. then for one hour at 20° C. This linker was inserted at the Xho I site of pMΔBB plasmid, yielding plasmid p2AB. Next, a second linker was made by annealing oligonucleotides 5′-CGAGCATG-3′ (SEQ. ID. NO. 18) and

5′-CTAGCATGCTCGAGCT-3′ (SEQ. ID. NO. 19). This linker was inserted between the Sac I and Nhe I site of pΔ2AB, yielding plasmid pMΔFM. Positive clones were sequenced as indicated above. Bacteria containing the pMΔFM plasmid were deposited on May 21, 2001 at the CNCM, under Accession Number I-2669.

To clone influenza HA sequences, viral genomic RNA was extracted from lung homogenates of A/PR/8/34(ma) infected mice using 5 M guanidium isothicanate and phenol using standard RNA extraction procedures. The resulting viral RNA was reverse transcribed into cDNA. Next, the HA coding sequences, including Bam HI sites before the initiation codon and after the terminating codon, were amplified by PCR with the PWO polymerase and the

5′-CTGGATCCAAAATGAAGGCAAACCT-3′ (SEQ. ID. NO. 20); and

5′-CAGGATCCTAGATGCATATTCTGCACTG-3′ (SEQ. ID. NO.21) oligonucleotides.

The resulting DNA fragment was cloned at the Bam HI site of plasmid pTG186, yielding plasmid pTG-HA8.

The coding sequences of the HA of the A/PR/8/34(ma) virus were then amplified by PCR using the oligonucleotides

5′-GAAAGGCAAACCTACTGGTCCTGTT-3′ (SEQ. ID. NO. 22) and

5′-CGTGCAGTCGACAGGATGCATATTCTGCACTGCAAAG-3′ (SEQ. ID. NO.23) using plasmid pTG-HA8 as a template. The oligonucleotides were designed so that the resulting DNA fragment could be digested by Sal I and cloned in frame between the klenow-destroyed Sac I site and the Nhe I site of plasmid p2ΔAB, yielding plasmid pMΔFM-HA. Positive clones were sequenced as indicated above. This plasmid contains a recombinant replicon cDNA, where the translation initiating AUG is followed by the HA sequences fused in frame with the 2A/2B autocatalytic cleavage site of Foot and Mouth Disease Virus (FMDV) followed by the CRE, the original Mengo virus 2A/2B cleavage site, and the remainder of the viral polyprotein (FIG. 1).

In Vitro Transcription of Plasmid DNA

The Mengo virus-derived plasmids were linearized with BamH I and transcribed using the Promega RiboMAX-T7 Large Scale RNA Production System (Promega #P1300) according to the manufacturers instructions. For in vivo studies, reaction mixtures were treated by RQ1 DNase (1.5 U/μg DNA, Promega #M6101) for 20 min at 37 C., extracted with phenol-chloroform, precipitated first in ammonium acetate-isopropyl alcohol, then in sodium acetate-isopropyl alcohol, via standard molecular biology techniques, and resuspended in endotoxin-free PBS (Life Sciences). For in vitro translation studies, reaction mixtures were processed the same way but precipitated once with ammonium acetate-isopropyl alcohol and resuspended in RNas free water.

Rabbit Reticulocyte Lysate In Vitro Translation

In vitro synthesized RNA (10 μg/ml) was translated in vitro using the Flex™ rabbit reticulocyte lysate system (Promega #L4540) supplemented with 0.8 mCi/ml of [³⁵S]-methionine (Amersham #SJ1515; 1000 Ci/mmol), 0.5 mM MgCl₂ and 100 mM KCl. Reaction mixtures were incubated for 3 hours at 30° C., treated with 100 μg/ml of RNase A in 10 mM EDTA for 15 minutes at 30° C., and analyzed by electophoresis on a 12% SDS polyacrylamide gel which were autoradiographed on Kodak X-OMAT film.

RNA Transfection

RNA transfection into HeLa cells was performed by electroporation using an Easyject plus electroporator (Equibio). Briefly, 16×10⁶ cells were trypsinized, washed twice with PBS, resuspended in 800 μl of ice-cold PBS and electroporated in the presence of 32 μg of RNA or DNA using a single pulse (240 V, 1800 μF, maximum resistance), in 0.4 cm electrode gap cuvettes. Cells were immediately transferred into DMEM complete medium with 2% FCS, distributed into eight 35 mm diameter tissue culture dishes, and incubated at 37° C., 5% CO₂.

Analysis of RNA Replication

At different time intervals post-transfection, cytoplasmic RNA was prepared using standard procedures (26). After denaturation in 1X SSC, 50% formamide, 7% formaldehyde for 15 min. at 65° C., the RNA samples were spotted onto a nylon membrane (Hybond N, Amersham #RPN203N) and hybridized with a ³²P-labelled RNA probe complementary to nucleotides 6022-7606 of Mengo virus RNA. Hybridizations were performed for 18 hours at 65° C. in a solution containing 6X SSC, 5X Denhardt solution and 0.1% SDS. The membranes were washed 3 times in a 2X SSC, 0.11% SDS solution at room temperature and another 3 times in a 0.1X SSC, 0.1% SDS solution at 65° C. Finally the membranes were exposed on a STORM™ 820 phosphorimager (Molecular Dynamics) and analyzed using the Image Quant program (Molecular Dynamics).

Analysis of GFP Expression in RNA-transfected Cells

HeLa cells were transfected as described above. Eight to twelve hours after transfection, cells were trypsinized, washed in PBS and fixed by incubation in 100 μl of PBS, 1% paraformaldehyde for 60 minutes at 4° C. Samples were then analyzed for fluorescence intensity on a FACScalibur fluorocytometer (Becton-Dickinson).

Analysis of Influenza NP Expression in RNA-transfected Cells

Influenza virus A/PR/8/34-infected or RNA/DNA-transfected cells were metabolically labeled with [³⁵S]-methionine (50 μCi/ml; Amersham; 1000 Ci/mmol) for 2 hours at times of peak expression. Peak expression times were determined by GFP expression studies in HeLa cells transfected with rMΔBB-GFP replicon RNA or pCI-GFP plasmid DNA. For RNA transfected cells, peak expression was observed between 6 and 9 hours post-transfection. For DNA transfected cells, peak expression was observed 20 hours post-transfection. For HeLa cells infected with A/PR/8/34 influenza virus, peak expression was observed at 20 hours post-infection. Next, cells were washed in PBS and lysed with 50 mM Tris-HCl pH 7.5, 150 mM NaCl, 1 mM EDTA, 1% NP40 and 0.5% Protease Inhibitor Cocktail (Sigma). Cell extracts were then immunoprecipitated overnight at 4° C. in RIPA buffer (50 mM Tris-HCl, 150 mM NaCl, 1 mM EDTA, 0.1% deoxycholate, 0.1% sodium dodecyl sulfate, 0.5% NP40 and 0.5% Protease Inhibitor Cocktail) in the presence of protein A sepharose beads (Amersham Pharmacia Biotech #17-0780-01) with rabbit antibodies raised against influenza A/PR/8/34 virus. The immunoprecipitates were washed in RIPA buffer, eluted in Laemmli sample buffer (50 mM Tris-HCl pH 6.8, 2% SDS, 5% β-mercaptoethanol, 20% glycerol) at 65° C., analyzed by SDS-PAGE, and visualized by autoradiography on Kodak X-OMAT film.

Analysis of the Expression of GFP Fusion Proteins in RNA-transfected Cells

Extracts of RNA/DNA transfected HeLa cells were immunoprecipitated and analyzed as described above for NP expression, but with rabbit antibodies raised against GFP (Invitrogen #46-0092).

Immunizations

C57BL/6 male mice (IFFA CREDO) 7 to 8 weeks of age, were injected intramuscularly (i.m.) with 100 μl of PBS (50 μl in each tibialis anterior muscle) containing either 50 μg of plasmid DNA or 25 μg of Mengo virus replicon RNA. Booster injections were administered via i.m. injection at 3 week intervals. DNA used for injection was prepared using the Nucleobond PC2000 kit (Nucleobond #740576), followed by extraction steps with triton X 114, then with phenol-chloroform. Samples were then tested for the absence of endotoxin (<100 U/mg) as measured with the QCL-1000 endotoxin kit (BioWhittaker #50-647U). RNA preparations were analyzed before and after injection by agarose gel electrophoresis to verify the absence of degradation.

Antibody Titer

Blood from mice was collected three weeks after the last injection. Serial dilutions of pooled serum samples were used to determine NP-specific antibody titers by ELISA using as antigen 0.5 μg of detergent-disrupted A/PR/8/34 virus per well. Briefly, 96-well ELISA plates (NUNC Maxisorp, #439454) were coated overnight at 4° C. with 0.5 μg of detergent-disrupted A/PR.8/34 virus in 0.2 M sodium carbonate, 0.2 M sodium bicarbonate, pH 9.6. Bound antibody was detected with a 1/2000 dilution of anti-mouse IgG(H+L) antibody conjugated to horseradish peroxidase (HRP) (Biosystems #B12413C) and visualized by adding TMB peroxidase substrate (KPL #50-76-00) as indicated by the supplier.

Titers were calculated as the reciprocal of the dilution of pooled serum that gave an optical density value at 450 nm equal to two times that of background levels. Pooled serum was prepared from a group of 4 or 5 mice.

Cytotoxicity Assay

Spleen cells were collected three weeks after the last immunization and seeded into upright T75 flasks at 2×10⁶ cells/ml in RPMI complete medium, supplemented with 10% FCS, 1.0 mM non-essential amino acids, 1 mM sodium pyruvate and 2.5% concanavalin A supernatant. Splenocytes were restimulated for 7 days with 10⁶ syngeneic spleen cells/ml, which had been pulsed for 3 hours at 37° C. with 10 μM NP366 peptide (ASNENMETM, Neosystem; SEQ. ID. NO. 24) in RPMI complete medium supplemented with 5% FCS, wed and irradiated (2500 rads). Cytotoxic Cavity of the restimulated effector cells was measured using a standard 4 hour ³¹Cr release cytotoxicity assay, essentially as described (9). EL4 and P815 target cells were pulsed or not with NP366 peptide (10 μM) during ⁵¹Cr labeling Spontaneous and maximal release of radioactivity were determined by incubating cells in medium alone or in 1% triton X-100, respectively. The percentage of specific ⁵¹Cr release was calculated as (experimental release—spontaneous release)/(maximal release—spontaneous release)×100.

IFNγ ELISPOT Assay

Spleen cells were collected three weeks after the last inoculation and analyzed for the presence of influenza or LCMV virus-specific CD8+ T cells in a standard IFNγ ELISPOT assay system. Briefly, spleen cells were stimulated for 20 hours with 1 μM influenza NP366 synthetic peptide (ASNENMETM, Neosystem; SEQ. ID. NO. 24) LCMV NP118-126 peptide (RPQASGVYM, Neosystem, SEQ. ID. NO. 25) and IL-2 (10 U/ml) in the presence of 5×10⁵ irradiated (2000 rads) syngenic spleen cells per well as feeder cells in 96-well Multiscreen HA nitrocellulose plates (Millipore), which had been coated with rat anti-mouse IFNγ antibodies (R4-6A2, Becton-Dickinson). Spots were revealed by successive incubations with biotintylated rat anti-mouse IFNγ antibodies (XMG1.2, Becton-Dickinson), alkaline phosphatase-conjugated streptavidin (Becton-Dickinson) and BCIP/NBT substrate (Sigma). The frequency of IFNγ-producing cells was determined by counting the number of spot-forming cells (SFC) in each well. Results were expressed as the number of SFC per 10⁵ spleen cells.

Challenge Infection of Mice with A/PR/8/34(ma) Virus

One or three weeks after the third immunization, C57BL/6 mice were lightly anaesthetized with 100 mg/kg of ketamine (Merial) and challenged intranasally with 100 pfu (0.1 LD₅₀) of A/PR/8/34(ma) virus in 40 μl of PBS. Mice were sacrificed seven days post-challenge. Lung homogenates were prepared and titered for virus on MDCK cell monolayers, in a standard plaque assay (36). Statistical analyses were performed on the log₁₀ of the viral titers measured for individual mice using the Student's independent t test, with the assumptions used for small samples (normal distribution of the variable, same for the populations to be compared).

Bacteria containing the plasmids pMΔBB and pMΔFM were deposited at the CNCM Institut Pasteur, 28, rue du Docteur Roux, 75724 Paris Cedex 15, France, as follows:

| | Accession | |
|---|---|---|
| Plasmid | Number | Deposit Date |
| pMΔBB (SEQ ID NO: 26) | I-2668 | May 21, 2001 |
| pMΔFM (SEQ ID NO: 27) | I-2669 | May 21, 2001 |
| pMΔBB-GFP-lcmvNP (SEQ ID NO: 28) | I-2879 | May 16, 2002 |

### EXAMPLE 1

### Production of Recombinant Replicons Derived from the Mengo Virus Genome

For the production of Mengo virus genome-derived replicons, plasmid vector pMΔBB was first constructed, in which the coding sequences of the L-P1-2A precursor of capsid proteins were substituted with a Sac I/Xho I polylinker and Mengo virus CRE, which was originally located in the VP2 capsid protein coding sequence (15). This substitution was done in a manner to maintain the sequences corresponding to an optimal 2A/2B autocatalytic cleavage site, consisting of the 19 C-terminal amino acids of 2A and the first amino acid of 2B (7) (FIG. 1). Specifically, plasmid pMC24, which contains the complete infectious cDNA of an attenuated strain of Mengo virus downstream of the T7 bacteriophage φ10 promoter, was deleted of nucleotides 737-3787, the L-P1-2A region that encodes the structural, L and 2A proteins. Deleted sequences were replaced by a Sac I, Xho I polylinker and a sequence encompassing Mengo virus CRE. Sequences encoding the 22 C-terminal amino acids of 2A that comprise the optimal sequence for in cis autocalalytic cleavage at the 2A/2B site were retained as described above The resulting plasmid, pMΔBB (SEQ ID NO: 26, 8017 base pairs), allows in vitro transcription with the T7 RNA polymerase of synthetic rMΔBB replicon RNA. The first base of SEQ ID NO: 26 corresponds to the first one of the replicon RNA, the BamH I site used for linearization of the plasmid before transcription is at position 4837 and the T7 promoter is from nucleotides 7999 to 8017 and 2G residues (nucleotides 8016 and 8017) are actually parts of the synthetic transcripts made from this plasmid with the T7 RNA polymerase.

The sequences for the GFP, the influenza NP or a GFP-NP fusion protein were then inserted into the polylinker of pMΔBB upstream of the CRE and the reconstituted 2A/2B cleavage site, in-frame with the rest of the sequences encoding the Mengo virus polyprotein yielding plasmid pMΔBB-GFP, pMΔBB-NP and pMΔBB-GFP-NP (FIG. 1).

For negative control purposes, plasmids pMΔXBB and pMΔXBB-GFP are similar to pMΔBB and pMΔBB-GFP, respectively, except these ΔX constructs do not contain the Mengo virus CRE (FIG. 1).

All plasmids described in this application were obtained in the laboratory using techniques known in the art. Their nucleotide sequences are known and available. They have been checked through complete sequencing of the inserts, when these have been obtained through PCR amplification.

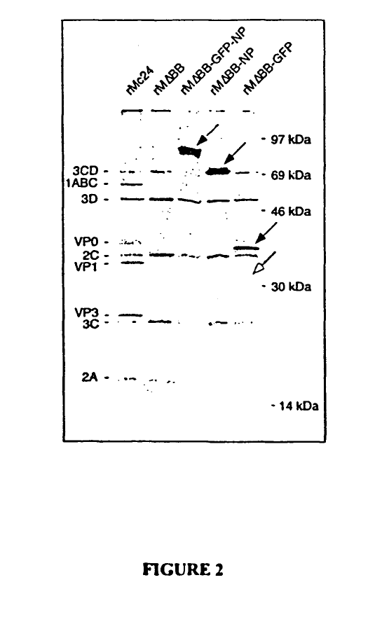

The recombinant RNAs, rMΔBB, rMΔBB-GFP, rMΔBB-NP and rMΔBB-GFP-NP, derived from in vitro transcription with 17 RNA polymerase of the pMΔBB, pMΔBB-GFP, pMΔBB-NP and pMΔBB-GFP-NP plasmid DNA, linearized with Bam HI, were translated in vitro in rabbit reticulocyte lysates. Translation products were analyzed by SDS-PAGE and visualized by autoradiography. As shown in FIG. 2, the replicon-encoded polyproteins were properly cleaved by the 3C protease to express the non-structural proteins necessary for RNA amplification, as evidenced by the end products of cleavage: such as the 2C, 3C, 3D and 3CD proteins. On the contrary, correct in cis cleavage of the reconstituted 2A/2B site was not observed for each of the rMΔBB derived replicons. The inventors anticipated that the foreign sequences would be expressed as a fusion protein with 7 linker encodod residues, the CRE encoded polypeptide (CREP, 44 amino-acids) and the last 22 residues of the 2A protein, enlarging the size of the foreign polypeptides by about 8 kD. For the recombinant rMΔBB-NP replicon, expression of the properly cleaved NP-CREP-2A* fusion protein would be revealed by the presence of a band with an expected molecular mass of 63 kDa, whereas a band of an approximate molecular mass of 70 kDa, or slightly heavier, was observed (FIG. 2). On the contrary, the GFP-CREP-2A* and GFP-NP-CREP-2A* fusion proteins migrated with a molecular mass similar to that expected (35 kDa and 89 kDa, respectively).

The inventors explain this apparent discrepancy between the expected size and the actual size of the NP protein made from the rMΔBB-NP replicon, in that the 2A/2B cleavage did not occur and, given the size of the 2B protein (151 amino-acids), an alternate cleavage occurred instead inside the 2B polypeptide, at approximately one third of its N-terminus. In this case, the NP related heterologous sequences encoded by the rMΔBB-NP vector were expressed as a NP-CREP-2A*-Δ2B fusion polypeptide. It is possible that the stretch of amino acids, encoded by the NP sequences and CRE and located before the cleavage site, forced the remainder of the 2A sequences to fold in a way which did not permit cleavage. The inventors currently have no explanation for the occurrence of an abnormal cleavage inside the 2B polypeptide, but alternate processing pathways have already been described for other picornaviruses, especially when one cleavage event of the processing cascade is blocked (4).

### EXAMPLE 2

### Replicative Characteristics of Mengo Virus Genome-derived Replicons, rMΔBB, rMΔBB-GFP, rMΔBB-NP, and rMΔBB-GFP-NP

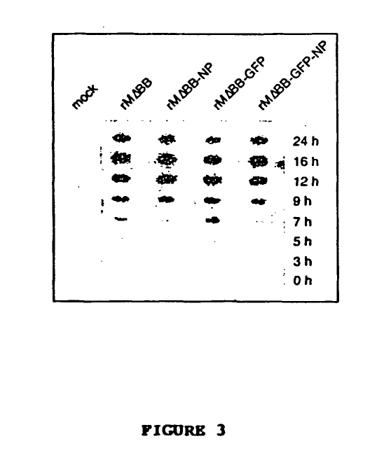

The inventors next determined if foreign sequences could be inserted into the Mengo virus genome without affecting replication of the RNA. Additionally, since the influenza NP has been shown to associate non-specifically with RNAs (14, 32), an interaction with the Mengo virus RNA could hypothetically affect overall replication efficiency. Therefore, synthetic RNA transcripts of rMΔBB, rMΔBB-FP, rMΔBB-NP and rMΔBB-GFP-NP were transfected into HeLa cells and total cytoplasmic RNA was extracted at various times post-transfection. Hybridization after slot blotting using a [³²P] radiolabeled riboprobe complementary to nucleotides 6022-7606 of Mengo virus RNA revealed efficient replication for all RNAs (FIG. 3). In the inventor's studies, cells were transfected by electroporation which was more efficient than the classic DEAE-dextran technique (>50% of the cells transfected). Under these conditions, all four RNA species induced a cytopathic effect (CPE), regardless of the presence or absence of capsid proteins, and resulted in the general destruction of the cell monolayer 24 hours post-transfection (data not shown). Taken together, these results illustrated that the insertion of foreign sequences, such as GFP or NP coding sequences, had no negative effect on RNA replication.

### EXAMPLE 3

### Expression of Green Fluorescent Protein by Recombinant Mengo Virus Derived Replicon

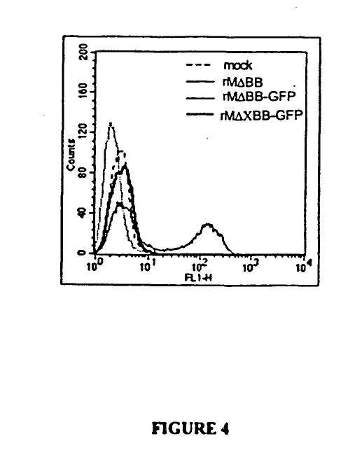

GFP expression was analyzed by cytofluorometry, monitoring the 530 nm fluorescence of cells transfected with Mengo virus-derived replicons. HeLa cells were mock transfected or transfected by electroporation with rMΔBB, rMΔBB-GFP or rMΔXBB-GFP replicon RNA. At 9 hours post-transfection, cells were trypsinized and then analyzed for fluorescence intensity on a FACScalibur fluorocytometer, as the period of GFP peak expression ranges from 7 to 12 hours for all the tested replicons according to results of preliminary experiments. As shown in FIG. 4, GFP expression could be detected in cells transfected with the rMΔBB-GFP but not in mock transfected cells or cells transfected with the empty vector rMΔBB. Interestingly, cells transfected with replicon rMΔXBB-GFP RNA did not show any fluorescence, confirming that Mengo virus CRE is required for RNA replication and demonstrating therefore that RNA replication is needed for significant expression of the foreign sequences. Thus, Mengo virus-derived recombinant replicons were shown to direct the efficient expression of the GFP in transfected cells.

### EXAMPLE 4

### Expression of Influenza Nucleoprotein by Recombinant Mengo Virus Derived Replicon

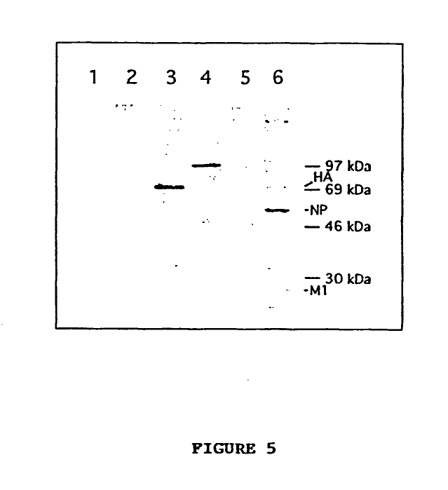

Nucleoprotein expression was analyzed by immunoprecipitation, with antibodies against A/PR/8/34 virus, of cytoplasmic extracts from cells transfected with Mengo virus-derived replicons or infected with A/PR/8/34 virus, as described in Methods. HeLa cells were transfected by electroporation with replicon RNA and at peak expression were metabolically labeled with [³⁵S]-methionine for 2 hours, according to results of preliminary experiments. Cytoplasmic extracts were prepared, and proteins were immunoprecipitated with polyclonal antibodies raised against influenza A/PR/8/34, analyzed by SDS-PAGE and visualized by autoradiography. As shown in FIG. 5, a protein with an apparent molecular mass of 70 kDa was specifically immunoprecipitated from extracts of cells transfected with rMΔBB-NP (lane 3). As expected, no immunoreactive proteins were detected from the mock transfected cells or from cells transfected with replicon RNA derived from the empty vector rMΔBB.

The NP fusion polypeptide expressed by the Mengo virus-derived replicon migrated with an apparent molecular mass of 70 kD (FIG. 5, lane 3), which is much higher than the molecular mass of 55 kD of the native form of NP expressed in A/PR/8/34 virus-infected cells (lane 6). As discussed above in Example 1, this difference in molecular mass accounted for the additional amino acid residues of the NP-CREP-2A* fusion protein and additional residues of the 2B protein, as it was observed in in vitro translation experiments. Again, this observation was consistent with the hypothesis that proteolytic processing at the 2A/2B site of the Mengo virus polyprotein did not occur and that an alternate cleavage site inside the 2B sequence was used instead. Interestingly, this did not affect overall replication efficiency of replicon RNA, suggesting that this alternate processing pathway could be part of the Mengo virus polyprotein processing cascade.

Transfection of HeLa cells with the recombinant replicon rMΔBB-GFP-NP (FIG. 5. lane 4) also resulted in high levels of NP-related protein expression. Again, no cleavage at the 2A/2B site seemed to occur as the NP-related material migrated with a molecular mass higher than expected (around 97 kDa instead of 89 kDa).

Thus Mengo virus-derived recombinant replicon were shown to direct the efficient expression in transfected cells of heterologous sequences of a size at least up to 2200 nucleotides.

### EXAMPLE 5

### Induction of a NP-specific CTL Response After Injection of Recombinant Mengo Virus Derived Replicon as Naked RNA

In order to establish the feasibility of using naked Mengo virus derived replicon injection for eliciting a heterospecific immune response, the inventors determined whether recombinant rMΔBB-NP injected as naked RNA was able to induce an NP-specific CTL response, specifically against NP's dominant H-2D^{b}-restricted epitope, NP366.

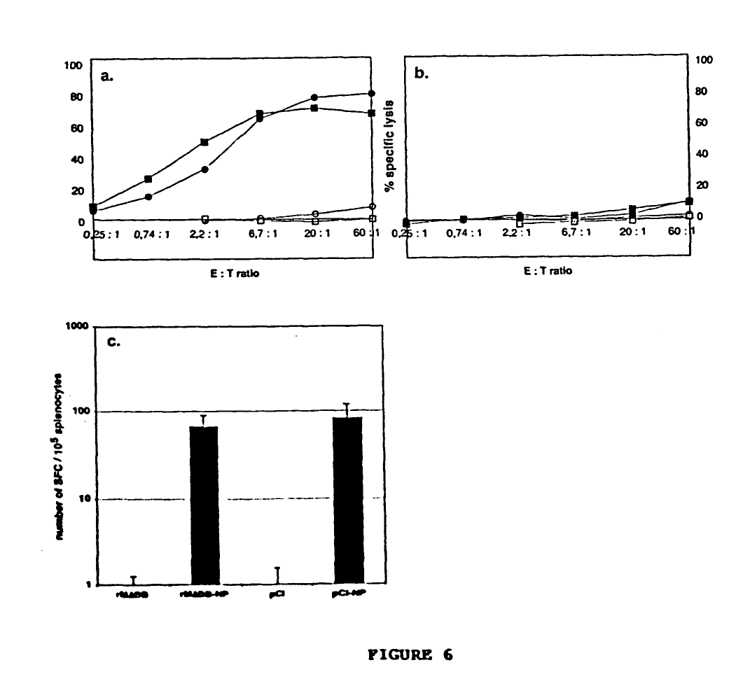

To this end, C57BL/6 mice were injected intramuscularly either twice with 25 μg of rMΔBB-NP naked RNA, at monthly intervals, or once with 50 μg of pCI-NP naked DNA as a positive control. This immunization schedule was defined according to previous experiments and based on the observation that one injection of plasmid DNA was sufficient to induce a detectable NP-specific CTL response at levels just below those obtained from mice having recovered from sublethal influenza A/PR/8/34(ma) infection (data not shown). Splenocytes from immunized mice were harvested 3 weeks after the last injection, stimulated in vitro with NP366 peptide and tested for cytolytic activity 7 days later in a classic chromium release assay, as described in Methods. Spleen cell cultures initiated from mice injected with rMΔBB-NP RNA or pCI-NP DNA specifically lysed syngeneic EL4 cells loaded with NP366 peptide (FIG. 6a). The CTL activity induced by rΔBB-NP replicon RNA was quite similar to the one induced by pCI-NP DNA and high (i.e., 60% to 70% specific lysis at an effector to target ratio of 6.7:1). In all cases, no lysis was observed with stimulated splenocytes from control naive mice or mice that were immunized with control vectors not bearing the NP sequences (FIG. 6, open symbols); nor was any lysis detected on syngeneic targets not charged with peptide (FIG. 6b). Finally, for all effector populations, lysis of allogenic P815 target cells (H-2⁴) remained at background levels regardless of whether or not they were incubated with peptide (data not shown), indicating that the cytolytic activity was H-2 restricted and thus likely to derive from class I restricted CD8⁺ T cell effectors.

Finally, the specific T cell responses induced by two i.m. injections of rMΔBB-NP RNA and pCI-NP DNA were quantified by the IFNγ ELISPOT assay. The frequency of IFNγ-producing cells was determined in response to in vitro stimulation of spleen cells from immunized mice with the influenza virus immunodominant NP366 peptide, as described in Materials and Methods. As shown in FIG. 6c, the T cell frequencies were remarkably high and in the same range (100 for 10⁵ splenocytes) for mice immunized with replicon RNA and plasmid DNA. As expected, less than 1 SFC per 10⁵ spleen cells were obtained in the absence of NP366 peptide or with spleen cells from mice immunized with empty vectors, serving as a mock control.

These findings thus showed that Mengo virus replicons were immunogenic when injected as naked RNA and were able to induce an heterospecific immunity against the inserted foreign sequences, such as those of the influenza NP.

### EXAMPLE 6

### Induction of NP Specific Antibody After Immunization with Recombinant rMΔBB-NP

In order to evaluate whether recombinant rMΔBB-NP injected as naked RNA was able to induce specific antibodies directed against influenza virus antigens, C57BL/6 mice were injected intramuscularly three times at three week intervals with 25 μg of rMΔBB-NP RNA or 50 μg of PCI-NP DNA as a positive control. Sera were collected three weeks after the last injection (1 or 2 for DNA, 2 for RNA). The specific anti-NP antibody response was examined by ELISA, as described in Materials and Methods.

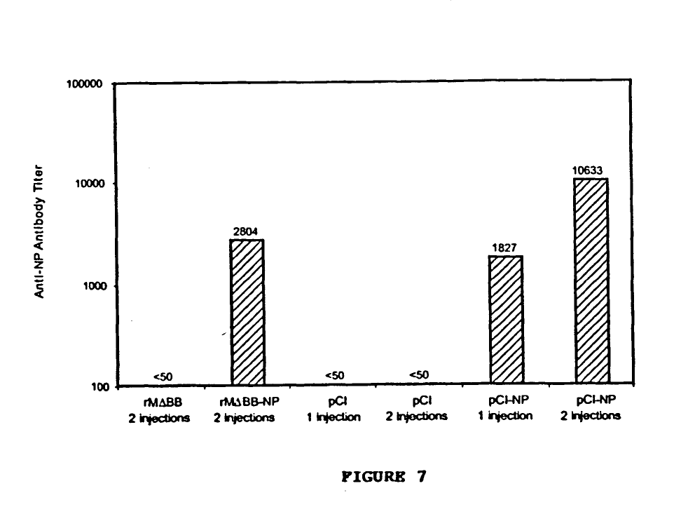

As shown in FIG. 7, two injections of 25 μg of naked rMΔBB-NP RNA induced serum antibodies against influenza NP. The NP-specific ELISA titers were slightly higher than those achieved by one injection of 50 μg of plasmid pCI-NP DNA but notably lower than those obtained after two injections of pCI-NP DNA.

As in Example 5. these findings showed that Mengo virus replicons were immunogenic when injected as naked RNA and were able to induce a heterospecific immune response against the inserted foreign sequence of the influenza NP. Taken together, Examples 5 and 6 demonstrate that Mengo virus replicons are able to induce both humoral (antibodies) and cellular (CTLs) immune response against an encoded heterologous protein.

### EXAMPLE 7

### Protective Immunity In Vivo

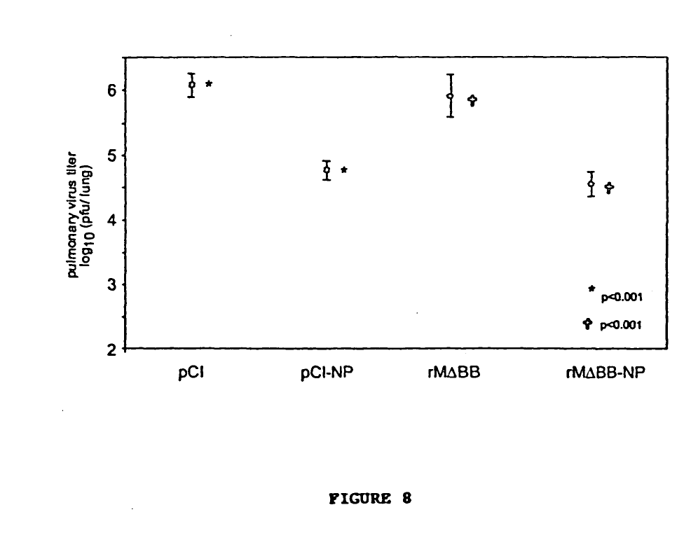

To show that the rMΔBB-NP can generate protective immunity in vivo, C57BL/6 mice (6 per group) were immunized 3 times at three week intervals with either 25 μg of rMΔBB or rMΔBB-NP replicon RNA or 50 μg of pCI or pCI-NP plasmid DNA. Three weeks after the last injection, mice were challenged with 10² pfu (0.1 LD50) of mouse-adapted A/PR/8/34 and viral titers in the lungs were determined 7 days post challenge infection. As shown in FIG. 8, Virus loads in mice injected with each NP-encoding vector were significantly lower than for mice injected with the corresponding empty vector (p<0.001; student's t test).

It is worth noting that although the drop in viral titer was moderate, which would correlate with the high virulence of the inventors' mouse-adapted viral strain (LD50 was 10³ pfu for C57BL/16 mice), the reduction in titer achieved with naked RNA immunization was as efficient as that obtained with the better described naked DNA immunization. This observation demonstrates that immune responses (most likely CTLs), induced by naked RNA immunization with Mengo virus-derived replicons, can contribute to protection against influenza by reducing pulmonary virus titer.

### EXAMPLE 8

### Production of the Recombinant rMΔFM Replicon Derived From the Mengo Virus Genome

In order to express foreign sequences in a more native form, the inventors explored the possibility of minimizing the size of vector sequences fused to the foreign ones. To achieve this, plasmid pMΔFM was constructed by the insertion of the sequences of the 2A/2B autocatalytic cleavage site of FMDV between the polylinker and CRE sequences of the pMΔBB encoded replicon (FIG. 1). In its optimal form, this cleavage site consists of 20 amino acids comprising the 19 C-terminal residues of the 2A protein and the first Proline of the 2B protein (7).

The resulting plasmid pMΔFM (8092 base pairs) corresponds to SEQ ID NO 27: the first base corresponds to the first one of the replicon RNA, the BamHI site used for linearization of the plasmid before transcription is a position 4912, the T7 promoter is from nucleotides 8074 to 8092 and 2G residues (nucleotides 8091 and 8092) are actually parts of the synthetic transcripts made from this plasmid with the T7 RNA polymerase.

Next, the sequences of the HA gene of the influenza A/PR/8/34(ma) virus were inserted between the Sac I and Nhe I sites of pMΔFM, immediately upstream of FMDV 2A sequences and in frame with the remaining polyprotein sequences, yielding plasmid pMΔFM-HA

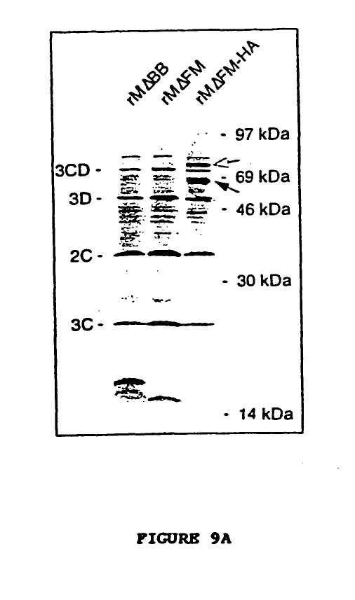

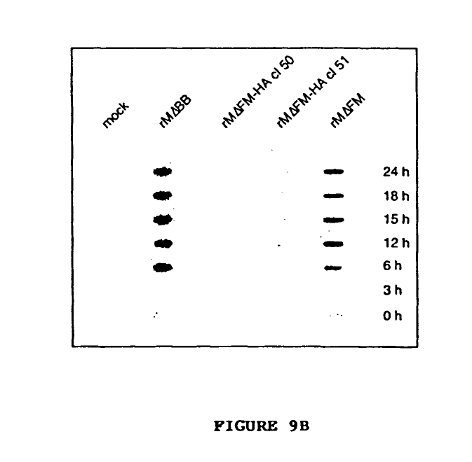

In order to verify that these constructs could be translated into polyproteins and cleaved into end products as predicted, corresponding linearized plasmids were transcribed in vitro and synthetic RNA were translated in rabbit reticulocytes lysates as described above. All replicons showed similar translation profiles of correctly cleaved end products, as evidenced by the presence of the 2C, 3C, 3D, and 3CD viral polypeptides (FIG. 9A).

In particular, correct in cis cleavage of the reconstituted FMDV 2A/2B site was observed for the recombinant replicon rMΔBB-HA; expression of the properly cleaved HA-2A* fusion protein, containing the 26 extra amino acids residues of the FMDV 2A protein (21 aa) and polylinker (5 aa), was hence revealed by the presence of a band with the expected molecular mass of 65 kDa (FIG. 9A). Interestingly, the presence of a band of higher molecular mass suggested that this cleavage was not 100% efficient in this in vitro translation assay.

For the corresponding parental replicon rMΔFM, such cleaved product, which would have appeared as a 3.4 kDa MCS-2A fusion protein, was not visible due to its small size, but a polypeptide of an apparent molecular mass of 16 kDa was present; this polypeptide could correspond to sequences spanning Mengo virus CRE, the last 22 residues of Mengo virus 2A and the N-terminus of 2B, suggesting that in this case the FMDV 2A/2B site was also cleaved whereas the original Mengo virus 2A/2B remained uncleaved, as was seen previously in the case of the rMΔBB and rMΔBB-NP replicons.

To test the replication efficiency of these second generation replicons, HeLa cells were transfected with synthetic RNAs by electroporation and at different time intervals post-transfection, cytoplasmic RNA was extracted and analyzed by Northern hybridization with a Mengo virus specific [³²P]-labeled riboprobe complementary to nucleotides 6022-7606 of the Mengo virus genome. As shown in FIG. 9B, the rMΔFM replicon did replicate as efficiently as its parent rMΔBB, indicating that the newly engineered 2A/2B cleavage had no adverse effect on RNA synthesis. On the other hand, the rMΔFM-HA recombinant replicon was not replication competent.

Because the HA present in the rMΔFM-HA replicon contained a SP and TM region, this finding may be similar to the case of replicons constructed from the genome of another picornavirus, the poliovirus. It was indeed found that the presence of a SP at the immediate N-terminus of a poliovirus replicon polyprotein abrogated replication of the corresponding RNA (1, 16). The inventors confirmed this observation recently by showing that the replication of a ΔP1 poliovirus replicon was abolished by the insertion of the complete sequences of the influenza HA, which is a glycosylated transmembrane protein (29). Moreover, the inventors demonstrated that it was possible to express the glycosylated sequences of the HA using replicons derived from the poliovirus genome and deleted of its P1 region, if these replicons were made dicistronic by the insertion of an heterologous IRES, such as the EMCV IRES, between the foreign sequences and the remaining P2P3 polyprotein sequences (29).

Therefore, dicistronic Mengo virus replicons can be constructed. This can be done in a first instance by the insertion of a foreign, viral or mammalian IRES between the Sac I/Xho I polylinker and the remaining polyprotein sequences of the pMΔBB plasmid. For example, such dicistronic Mengo virus replicons can be constructed by inserting the foreign IRES of equine rhinitis virus type A or type B, because both of these IRESes compete efficiently for translation factors with the IRES of EMCV virus, which is the prototype of the cardiovirus genus (38). Such dicistronic Mengo virus replicons can replicate and express glycosylated foreign polypeptides, as it was demonstrated by the inventors' previous work with dicistronic poliovirus replicons. For example, the influenza HA sequences can be inserted in one of these new dicistronic Mengo virus replicons.

These new dicistronic Mengo virus replicons will allow the expression of foreign antigens or proteins of interest, when glycosylation is a key parameter of the antigenicity or biological activity of the polypeptide. For example, Mengo virus dicistronic replicons can be used to express either viral antigens, such as the HBs antigen of the Hepatitis B virus or the envelope glycoprotein of the Human Immunodeficiency Virus, or cancer antigens, such as surface antigens of human tumor cells.

The Mengo virus rMΔFM replicon vector can also be used to direct the native expression of non-glycosylated foreign protein in transfected cells, as it was observed in rabbit reticulocyte lysates.

### EXAMPLE 9

### Expression of Other Antigens, LCMV Nucleoprotein (NP) or LCMV NP118-126 Epitope by Mengo Virus Replicons

In order to show that Mengo virus-derived replicons inoculated as naked RNA were able to induce heterospecific immune responses against other antigens, the inventors constructed the rMΔBB-GFP-1cmvNP and rMΔBB-GFP-NP118 replicons. These replicons encode respectively the NP and the NP118-126 H2^{d}-restricted immunodominant epitope of LCMV as fusion proteins with GFP.

To achieve this, the plasmid pMΔBB-GFP-1cmvNP (SEQ ID NO: 28, 10417 base pairs) was constructed as described in materials and methods. The first base of SEQ ID NO: 28 corresponds to the first one of the replicon RNA. The BamHI site used for linearization of the plasmid before transcription is at position 7237. The T7 promoter is from nucleotides 10399 to 10417 and 2G residues (nucleotides 10416 and 0417) are actually parts of the synthetic transcripts made from this plasmid with the T7 RNA polymerase.

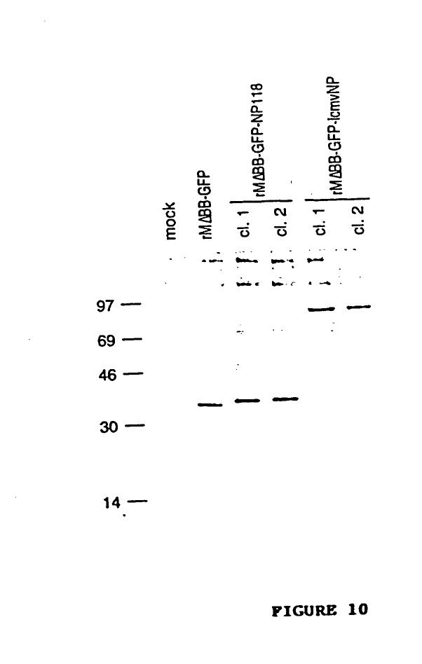

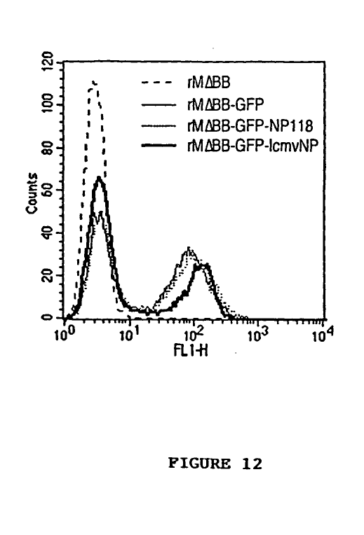

Next, expression of the LCMV NP as a fusion polypeptide with GFP was revealed by the presence of a band with an expected molecular mass of 97 kDa in cytosolic extracts of HeLa cells, which had been electroporated with rMΔBB-GFP-1cmvNP replicon RNA (FIG. 10). GFP expression could also be evidenced by cytofluorometry, monitoring the 530 nm fluorescence of HeLa cells transfected with the replicon (FIG. 12). Similarly, expression of the NP118-126 LCMV epitope as a 15 amino acid precursor (NP116-130, roughly 1.7 kDa) was detected as a fusion protein, slightly heavier than GFP (35 kDa). This indicated that the recombinant rMΔBB-GFP-1cmvNP and rMΔBB-GFP-NP118 RNAs did replicate and permitted the synthesis of the inserted sequences as was the case for the parental rMΔBB-GFP replicon described above. Furthermore, together with Example 3, it showed that GFP expression could be easily used as a marker for RNA replication of suitable Mengo virus-derived replicons.

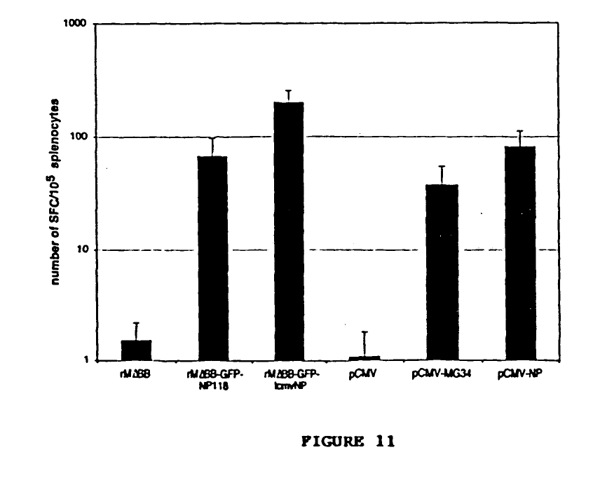

Last, BALB/c mice were injected i.m. twice with 25 μg of rMBB-GFP, rMΔBB-GFP-1cmvNP, or rMΔBB-GFP-NP118 naked RNA or with 50 μg of pCMV-NP or pCMV-MG34 (40) naked DNA as a positive control. The frequency of IFNγ-producing cells was determined by the IFNγ ELISPOT assay in response to in vitro stimulation of spleen cells from immunized mice with the LCMV immunodominant NP118-126 peptide, as described in Materials and Methods. As shown in FIG. 11, both rMΔBB-GFP-1cmvNP and rMΔBB-GFP-NP118 replicons induced high frequencies of LCMV-specific T cells (70 to 200 for 10⁵ splenocytes). Interestingly, these frequencies were slightly higher than those observed after genetic immunization with plasmid DNA

In conclusion, these findings showed that Mengo virus replicons are versatile tools for inducing heterospecific immune responses, as they can express in an immunogenic form either full-length foreign antigens or short relevant peptides corresponding to foreign epitopes.

Having now fully described the invention, it will be appreciated by those skilled in the art that the invention can be performed within a range of equivalents and conditions without departing from the spirit and scope of the invention and without undue experimentation. In addition, while the invention has been described in light of certain embodiments and examples, the inventors believe that it is capable of further modifications. This application is intended to cover any variations, uses, or adaptations of the invention which follow the general principles set forth above.

All references, manuals, patents, and patent applications cited herein are incorporated by reference in their entirety.

References:

1. Ansardi, D. C., Z Moldoveanu, D. C. Porter, D. E. Walker, R M. Conry, A. F. LoBuglio, S. McPherson, and C. D. Morrow 1994. Characterization of poliovirus replicons encoding carcinoembryonic antigen. Cancer Res. 54:6359-64.
2. Bot, A., S. Bot4 A. Garcia-Sastre, and C. Bona 1996. DNA immunization of newborn mice with a plasmid-expressing nucleoprotein of influenza virus. Viral Immunol. 9:207-10.
3. Choi, W. S., R. Pal-Ghosh, and C. D. Morrow 1991. Expression of human immunodeficiency virus type I (HIV-1) gag, pol, and env proteins from chimeric HIV-1-poliovirus minireplicons. J. Virol. 65:2875-83.
4. Cohen, L., K. M. Kean, M. Girard, and S. Van der Werf 1996. Effects of P2 cleavage site mutations on poliovirus polyprotein processing. Virology. 224:34-42.
5. Conry, R. M., A. F. LoBuglio, M. Wright, L. Sumerel, M. J. Pike, F. Johanning, R. Benjamin, D. Lu, and D. T. Curiel 1995. Characterization of a messenger RNA polynucleotide vaccine vector. Cancer Res. 55:1397-400.
6. Dalemans, W., A. Delers, C. Delmelle, F. Denamur, R. Meykens, C. Thirart, S. Veenstra M. Francotte, C. Bruck, and J. Cohen 1995. Protection against homologous influenza challenge by genetic immunization with SFV-RNA encoding Flu-HA. Annals of the New York Academy of Sciences. 772:255-6.
7. Donnelly, M. L., D. Gani, M. Flint, S. Monaghan, and M. D. Ryan 1997. The cleavage activities of aphthovirus and cardiovirus 2A proteins. J. Gen. Virol. 78:13-21.
8. Duke, G. M, and A. C. Palmenberg 1989. Cloning and synthesis of infectious cardiovirus RNAs containing short, discrete Poly(C) tracts. J. Virol 63:1822-1826.
9. Escriou, N., C. Leclerc, S. Gerbaud, M. Girard, and S. van der Werf 1995. Cytotoxic T cell response to Mengo virus in mice: effector cell phenotype and target proteins. J. Gen. Virol. 76:1999-2007.
10. Fleeton, M. N., M. Chen, P. Berglund, G. Rhodes, S. E. Parker, M. Murphy, G. J. Atkins, and P. Liljeström 2001. Self-replicating RNA vaccines elicit protection against Influenza A Virus, Respiratory Syncytial Virus, and a Tickborne Encephalitis Virus. J. Infect. Dis 183:1395-8.
11. Frolov, I., T. A. Hoffman, B. M. Pragai, S. A. Dryga, H. V. Huang, S. Schlesinger, and C. M. Rice 1996. Alphavirus-based expression vectors: strategies and applications. Proc. Natl. Acad. Sci. USA. 93:11371-7.
12. Kaplan, G., and V. R. Racaniello 1988. Construction and characterization of poliovirus subgenomic replicons. J. Virol 62:1687-96.
13. Kieny, M. P., G. Rautmann, D. Schmitt, K. Dott, S. Wain-Hobson, M. Alizon, M. Girard, S. Chamaret, A. Laurent, L. Montagnier, and J. P. Lecocq 1986. AIDS virus env protein expressed from a recombinant vaccinia virus. Biotechnology. 4:790-795.
14. Kingsbury, D. W., I. M. Jones, and K. G. Murti 1987. Assembly of influenza ribonucleoprotein in vitro using recombinant nucleoprotein. Virology. 156:396-403.
15. Lobert, P. E., N. Escriou, J. Ruelle, and T. Michiels 1999. A coding RNA sequence acts as a replication signal in cardioviruses. Proc. Natl. Acad. Sci. USA. 96:11560-5.
16. Lu, H. H., L. Alexander, and E. Wimmer 1995. Construction and genetic analysis of dicistronic polioviruses containing open reading frames for epitopes of human immunodeficiency virus type 1 gp120. J. Virol. 69:4797-806.
17. Martinon, F., S. Krishnan, G. Lenzen, R. Magno, E. Gomard, J.-G. Guillet, J.-P. Levy, and P. Meulien 1993. Induction of virus-specific cytotoxic T lymphocytes in vivo by liposome-entrapped mRNA. Eur. J. Immunol. 23:1719-1722.
18. Moldoveanu, Z., D. C. Porter, A. Lu, S. McPherson, and C. D. Morrow 1995. Immune responses induced by administration of encapsidated poliovirus replicons which express HIV-1 gag and envelope proteins. Vaccine. 13:1013-22.
19. Nichols, W. W., B. J. Ledwith, S. V. Manarn and P. J. Troilo 1995. Potential DNA vacine integration into host cell genome. Annals of the New York Academy of Sciences. 772:30-9.
20. Oukka, M., J. C. Manuguerra, N. Livaditis, S. Tourdot, N. Riche, I. Vergnon, P. Cordopatis, and K. Kosmatopoulos 1996. Protection against lethal viral infection by vaccination with nonimmunodominant peptides. J. Immunol. 157:3039-45.
21. Percy, N., W. S. Barclay, M. Sullivan, and J. W. Almond 1992. A poliovirus replicon containing the chloramphenicol acetyltransferase gene can be used to study the replication and encapsidation of poliovirus RNA. J. Virol. 66:5040-6.
22. Pogulis, R J., A. N. Vallejo, and L. R. Pease 1996. In vitro recombination and mutagenesis by overlap extension PCR Methods Mol Biol. 57:167-76.
23. Porter, D. C., J. Wang, Z. Moldoveanu, S. McPherson, and C. D. Morrow 1997. Immunization of mice with poliovirus replicons expressing the C-fragment of tetanus toxin protects against lethal challenge with tetanus toxin. Vaccine. 15:257-64.
24. Pushko, P., M. Parker, G. V. Ludwig, N. L. Davis, R. E. Johnston, and J. F. Smith 1997. Replicon-helper systems from attenuated Venezuelan equine encephalitis virus: expression of heterologous genes in vitro and immunization against heterologous pathogens in vivo. Virology. 239:389-401.
25. Qiu, P., P. Ziegelhoffer, J. Sun, and N. S. Yang 1996. Gene gun delivery of mRNA in situ results in efficient transgene expression and genetic immunization. Gene Therapy. 3:262-8.
26. Sambrook, J., E. F. Fritsch, and T. Maniatis 1989. Molecular cloning: a laboratory manual, 2nd ed, vol. 1. Cold Spring Harbor Laboratory Press, N.Y.
27. Ulmer, J. B., J. J. Donnelly, S. E. Parker, G. H. Rhodes, P. L. Felgner, V. J. Dwarki, S. H. Gromkowsli, R. R. Deck, C. M. DeWitt, A. Friedman, L. A. Hawe, K. R. Leander, D. Martinez, K. C. Perry, J. W. Shiver, D. L Montgomery, and M. A. Liu 1993. Heterologous protection against influenza by injection of DNA encoding a viral protein. Science 259:1745-1749.
28. J. B., T. M. Fu, R. R. Deck, A. Friedman, L. Guan, C. DeWitt, X. Liu, S. Wang. A . Liu, J. J. Donnelly, and M. J. Caulfield 1998. Protective CD4+ and CD8+ T cells against influenza virus induced by vaccination with nucleoprotein DNA. J. Virol. 72:5648-53.
29. Vignuzzi, M., S. Gerbaud, S. van der Werf. and N. Escriou 2002 Expression of a membrane-anchored glycoprotein, the Influenza hemagglutinin, by dicistronic replicons derived from the poliovirus genome. J. Virol. 76:5285-90.
30. Vignuzzi, M., S. Gerbaud, S. van der Werf, and N. Escriou 2001. Naked RNA immunization with replicons derived from the poliovirus and Semliki Forest virus genomes for the generation of a cytotoxic T cell (CTL) response against the Influenza A virus nucleoprotein. J. Gen. Virol. 82:1737-47.
31. Wolff, J. A., J. J. Ludtke, G. Acsadi, P. Williams, and A. Jani 1992. Long-term persistence of plasmid DNA and foreign gene expression in mouse muscle. Human Molecular Genetics. 1:363-9.
32. Yamanaka, K, A. Ishihama, and K. Nagata 1990. Reconstitution of influenza virus RNA-nucleoprotein complexes structurally resembling native viral ribonucleoprotein cores. J. Biol. Chem. 265:11151-5.
33. Ying, H., T. Z., Zaks, R F. Wang, K. R. Irvine, U. S. Kammula, F. M. Marincola, W. W. Leitner, and N. P. Restifo 1999. Cancer therapy using a self-replicating RNA vaccine. Nature Medicine. 5:823-7.
34. Zhou, X., P. Berglund, G. Rhodes, S. E. Parker, M. Jondal, and P. Liljeström 1994. Self-replicating Semliki Forest virus RNA as recombinant vaccine. Vaccine. 12:1510-1514.
35. Kurth, R. 1995. Risk potential of the chromosomal insertion of foreign DNA. Ann. N.Y. Acad. of Sci. 772:140-51.
36. Manuguerra, J. C. and C. Hannoun 1999. Influenza and other Viral Respiratory Diseases, surveillance and laboratory diagnosis. Edited by the Institut Pasteur. ISBN 2-901320-28-7.
37. Hoerr, I., R. Obst, H. G. Rammensee, and G. Jung 2000. In vivo application of RNA leads to induction of specific cytotoxic T lymphocytes and antibodies. Eur. J. Immunol. 30:1-7.
38. Hinton T. M. and B. S. Crabb 2001. The novel picornavirus Equine rhinitis B virus contains a strong type II internal ribosomal entry site which functions similarly to that of Encephalomyocarditis virus. J. Gen. Virol. 82:227-69.
39. Yokoyama, M. J. Zhang, and J. L. Whitton 1995. DNA immunization confers protection against lethal lymphocytic choriomeningitis virus infection. J. Virol. 69:2684-88.
40. Rodriguez, F. L. L. An, S. Harkins, J. Zhang, M. Yokouama, G. Widera, J. T. Fuller, C. Kincaid, I. L. Campbell, and J. L. Whitton 1998. DNA immunization with minigenes: low frequency of memory cytotoxic T lymphocytes and inefficient antiviral protection are rectified by ubiquitination. J. Virol. 72:5174-81.

1. A self-replicating recombinant positive strand RNA molecule of a viral genome of an RNA virus, wherein the RNA molecule comprises:
a) RNA sequence encoding the non-structural proteins of the RNA virus;
b) viral non-encoding RNA sequences necessary for viral replication; and
(c) RNA sequence encoding a heterologous protein or fragment of a heterologous protein.

2. A self-replicating recombinant positive strand RNA molecule of a viral genome of an RNA virus, wherein the RNA molecule comprises:
(a) RNA sequence encoding the non-structural proteins of the RNA virus;
(b) viral non-encoding RNA sequences necessary for viral replication;
wherein the RNA sequence in a) and/or the viral non-encoding RNA sequences in b) are either in mutated or truncated forms, and
(c) RNA sequence encoding a heterologous protein or fragment of a heterologous protein.

3. The self-replicating recombinant positive strand RNA molecule according to claims 1 or 2, wherein the RNA virus is in the genus of Cardiovirus or Aphtovirus.

4. The self-replicating recombinant positive strand RNA molecule of claim 3, wherein the RNA virus is a Mengo virus.

5. The self-replicating recombinant positive strand RNA molecule of claim 4 further comprising the Cis-acting Replication Element (CRE) of the Mengo virus VP2 gene.

6. The self-replicating recombinant positive strand RNA molecule of claim 4 further comprising the Cis-acting Replication Element (CRE) of the Theiler's virus VP2 gene.

7. The self-replicating recombinant positive strand RNA molecule according to claims 1 or 2, wherein the heterologous protein is chosen from a biologically active protein, a reporter protein, a cytotoxic protein, a protein of a pathogen, or a protein of a tumor.

8. The self-replicating recombinant positive strand RNA molecule of claim 7, wherein the reporter protein is green fluorescent protein.

9. The self-replicating recombinant positive strand RNA molecule of claim 7, wherein the protein of a pathogen is influenza nucleoprotein or influenza hemagglutinin.

10. The self-replicating recombinant positive strand RNA molecule according to claims 1 or 2, wherein the heterologous protein fragment is an antigen or epitope of said heterologous protein.

11. A vaccine comprising at least one self-replicating recombinant positive strand RNA molecule according to claims 1 or 2, and a pharmaceutically acceptable carrier.

12. The vaccine of claim 11, wherein the self-replicating recombinant positive strand RNA molecule is naked RNA.

13. The vaccine of claim 11, wherein the self-replicating recombinant positive strand RNA molecule is encapsidated.

14. The vaccine according to claims 11, wherein the pharmaceutically acceptable carrier is chosen from water, petroleum oil, animal oil, vegetable oil, peanut oil, soybean oil, mineral oil, sesame oil, saline solutions, aqueous dextrose, glycerol solutions, polycationic particles, protein particles, protamine particles, liposomes, and gold particles.

15. A method of inducing a protective immune response in a host comprising:
(a) at least one self-replicating recombinant positive strand RNA molecule of claims 1 or 2 in a pharmaceutically acceptable carrier, and
b) immunizing the host with the preparation of step (a).

16. A method of inducing an immune response in a host according to claim 15, wherein the self-replicating recombinant positive strand RNA molecule of step (a) is prepared in naked form.

17. A method of inducing an immune response in an a host according to claim 15, wherein the self-replicating recombinant positive strand RNA molecule of step (a) is an encapsidated RNA.

18. The method according to claim 15, wherein the pharmaceutically acceptable carrier is chosen from water, petroleum oil, animal oil, vegetable oil, peanut oil, soybean oil, mineral oil, sesame oil, saline solutions, aqueous dextrose, glycerol solutions, polycationic particles, protein particles, protamine particles, liposomes, and gold particles.

19. The method according to claim 15, wherein the host is a human, a pig, a dog, a cat, a cow, a chicken, a mouse, or a horse.

20. A DNA molecule that encodes a self-replicating recombinant positive strand RNA molecule of a viral genome of an RNA virus, wherein the RNA molecule comprises:
(a) RNA sequence encoding the non-structural proteins of the RNA virus;
(b) viral non-encoding RNA sequences necessary for viral replication; and
(c) RNA sequence encoding a heterologous protein or fragment of a heterologous protein.

21. A DNA molecule that encodes a self-replicating recombinant positive strand RNA molecule of a viral genome of an RNA virus, wherein the RNA molecule comprises:
(a) RNA sequence encoding the non-structural proteins of the RNA virus;
(b) viral non-encoding RNA sequences necessary for viral replication; wherein the RNA sequence in a) and/or the viral non-encoding RNA sequences in b) are either in mutated or truncated forms, and
(c) RNA sequence encoding a heterologous protein or fragment of a heterologous protein.

22. The DNA molecule according to claims 20 or 21, wherein the RNA virus is in the genus of Cardiovirus or Aphtovirus.

23. The DNA molecule according to claim 22, wherein the RNA virus is a Mengo virus.

24. The DNA molecule of claim 23, further encoding RNA comprising the Cis-acting Replication Element (CRE) of the Mengo virus VP2 gene.

25. The DNA molecule of claim 23, further encoding RNA comprising the Cis-acting Replication Element (CRE) of the Theiler's virus VP2 gene.

26. The DNA molecule according to claims 20 or 21, wherein the heterologous protein is chosen from a biologically active protein, a reporter protein, a cytotoxic protein, a protein of a pathogen, or a protein of a tumor.

27. The DNA molecule of claim 26, wherein the reporter protein is green fluorescent protein.

28. The DNA molecule of claim 26, wherein the protein of a pathogen is influenza nucleoprotein or influenza hemagglutinin.

29. The DNA molecule of claim 26, wherein the heterologous protein fragment is an antigen or epitope of said heterologous protein.

30. The DNA molecule of claim 26, further comprising a suitable cloning vector.

31. A DNA molecule comprising the sequence of SEQ. ID. NO. 26 (deposited at CNCM, Institut Pasteur, 28 rue du Docteur Roux, 75724 Paris Cedex 15, France, on May 21, 2001, under Accession No.1-2668) or a fragment thereof, and DNA sequence encoding a heterologous protein or fragment of a heterologous protein in an expressible form.

32. A DNA molecule comprising the sequence of SEQ. ID. NO. 26 (deposited at CNCM, Institut Pasteur, 28 rue du Docteur Roux, 75724 Paris Cedex 15, France, on May 21, 2001, under Accession No. I-2668) either in a mutated or truncated form or a fragment thereof and DNA sequence encoding a heterologous protein or fragment of a heterologous protein in an expressible form.

33. The DNA molecule according to claims 31 or 32, wherein the heterologous protein is chosen from a biologically active protein, a reporter protein, a cytotoxic protein, a protein of a pathogen, or a protein of a tumor.

34. The DNA molecule according to claim 33, wherein the reporter protein is green fluorescent protein.

35. The DNA molecule according to claim 33, wherein the protein of a pathogen is influenza nucleoprotein or influenza hemagglutinin.

36. The DNA molecule according to claims 31 or 32, wherein the heterologous protein fragment is an antigen or epitope of said heterologous protein.

37. A DNA molecule comprising the sequence of SEQ. ID. NO. 27 (deposited at the CNCM, Institut Pasteur, 28 rue du Docteur Roux, 75724 Paris Cedex 15, France, on May 21, 2001, under Accession No. I-2669) or a fragment thereof and DNA sequence encoding a heterologous protein or fragment of a heterologous protein.

38. A DNA molecule comprising the sequence of SEQ. ID. NO. 27 (deposited at the CNCM, Institut Pasteur, 28 rue du Docteur Roux, 75724 Paris Cedex 15, France, on May 21, 2001, under Accession No. I-2669) either in a mutated or truncated form or a fragment thereof and DNA sequence encoding a heterologous protein or fragment of a heterologous protein in an expressible form.

39. The DNA molecule according to claims 37 or 38, wherein the heterologous protein is chosen from a biologically active protein, a reporter protein, a cytotoxic protein, a protein of a pathogen or a protein of a tumor.

40. The DNA molecule according to claim 39, wherein the protein of a pathogen is influenza nucleoprotein or influenza hemagglutinin.

41. The DNA molecule according to claims 37 or 38, wherein the heterologous protein fragment is an antigen or epitope of said heterologous protein.

42. A method of inducing a protective immune response in a host comprising:
(a) providing a preparation comprising at least one DNA molecule of claims 20 or 21 in a pharmaceutically acceptable carrier; and
(b) immunizing the host with the preparation of step (a).

43. A method of inducing a protective immune response in a host according to claim 42, wherein the DNA molecule is naked DNA.

44. A method of inducing a protective immune response in a host according to claim 42, wherein the DNA molecule is encapsidated.

45. A therapeutic composition comprising at least a DNA molecule according to claims 20 or 21 or a self-replicating recombinant positive strand RNA molecule according to claims 1 or 2 in an acceptable medium.

46. A therapeutic kit comprising at least a DNA molecule according to claims 20 or 21 or a self-replicating recombinant positive strand RNA molecule according to claims 1 or 2 in an acceptable medium.

47. A method for modulating the immune response in a host comprising:
(a) preparing at least one molecule selected from the DNA molecule of any of claims 20 or 21 and the self-replicating recombinant positive strand RNA molecule of any of claims 1 or 2 and in a pharmaceutically acceptable carrier; and
(b) immunizing the host with the preparation of step (a).

48. The method of claim 47, wherein the pharmaceutically acceptable carrier is chosen from water, petroleum oil, animal oil, vegetable oil, peanut oil, soybean oil, mineral oil, sesame oil, saline solutions, aqueous dextrose, glycerol solutions, polycationic particles, protein particles, protamine particles, liposomes, and gold particles.

49. The method of claim 47, wherein the host is a human, a pig, a dog, a cat, a cow, a chicken, a mouse, or a horse.

50. A method for improving the immunogenicity of a self-replicating recombinant positive strand RNA molecule of a viral genome of an RNA virus by producing an encapsidated self-replicating recombinant positive strand RNA molecule of a viral genome of an RNA virus comprising:
(a) transfecting the self-replicating recombinant positive strand RNA molecule of any of claims 1 or 2 and or the DNA molecule of any of claims 20 or 21 into cells expressing the P1 precursor of capsid proteins;
(b) preparing the encapsidated self-replicating recombinant positive strand RNA molecule from the transfected cells; and
(c) immunizing a host with the preparation of step (b).

51. A method for improving the immunogenicity of a self-replicating recombinant positive strand RNA molecule of a viral genome of an RNA virus comprising:
(a) condensing the self-replicating recombinant positive strand RNA molecule of any of any of claims 1 or 2; and
(b) immunizing a host with the condensed RNA molecule of step (a).

52. A DNA molecule, comprising the sequence of SEQ. ID. NO. 28 (deposited at CNCM, Institut Pasteur, 28 rue du Docteur Roux, 75724 Paris Cedex 15, France, on May 16, 2002, under Accession No. I-2879).

53. The DNA molecule according to claims 36, wherein the epitope of said heterologous protein is the NP118-126 epitope of the lymphocytic choriomeningitis virus nucleoprotein.

54. The DNA molecule according to claim 41, wherein the epitope of said heterologous protein is the NP118-126 epitope of the lymphocytic choriomeningitis virus nucleoprotein.


SEQUENCE LISTING (28 sequences)

SEQ ID NO: 1 - DNA, 9 bp, Artificial Sequence
  Description of Artificial Sequence Synthetic oligonucleotide
gagctcgag

SEQ ID NO: 2 - DNA, 13 bp, Artificial Sequence
  Description of Artificial Sequence Synthetic oligonucleotide
tcgaggctagctt

SEQ ID NO: 3 - DNA, 11 bp, Artificial Sequence
  Description of Artificial Sequence Synthetic oligonucleotide
cgaagctagcc

SEQ ID NO: 4 - DNA, 31 bp, Artificial Sequence
  Description of Artificial Sequence Synthetic oligonucleotide
gctgagctcatggtgagcaagggcgaggagc

SEQ ID NO: 5 - DNA, 31 bp, Artificial Sequence
  Description of Artificial Sequence Synthetic oligonucleotide
gcagagctccttgtacagctcgtccatgccg

SEQ ID NO: 6 - DNA, 20 bp, Artificial Sequence
  Description of Artificial Sequence Synthetic oligonucleotide
tctccacaggtgtccactcc

SEQ ID NO: 7 - DNA, 29 bp, Artificial Sequence
  Description of Artificial Sequence Synthetic oligonucleotide
cacatcctggggtccattccggtgcgaac

SEQ ID NO: 8 - DNA, 30 bp, Artificial Sequence
  Description of Artificial Sequence Synthetic oligonucleotide
accggaatggaccccaggatgtgctctctg

SEQ ID NO: 9 - DNA, 20 bp, Artificial Sequence
  Description of Artificial Sequence Synthetic oligonucleotide
gtcccatcgagtgcggctac

SEQ ID NO: 10 - DNA, 36 bp, Artificial Sequence
  Description of Artificial Sequence Synthetic oligonucleotide
cggaattctcgagatggcgtctcaaggcaccaaacg

SEQ ID NO: 11 - DNA, 37 bp, Artificial Sequence
  Description of Artificial Sequence Synthetic oligonucleotide
gcgaattctcgagattgtcgtactcctctgcattgtc

SEQ ID NO: 12 - DNA, 37 bp, Artificial Sequence
  Description of Artificial Sequence Synthetic oligonucleotide
cggaattctcgagatgtccttgtctaaggaagttaag

SEQ ID NO: 13 - DNA, 33 bp, Artificial Sequence
  Description of Artificial Sequence Synthetic oligonucleotide
gcgaattctcgagtgtcacaacatttgggcctc

SEQ ID NO: 14 - DNA, 54 bp, Artificial Sequence
  Description of Artificial Sequence Synthetic oligonucleotide
tcgaagctagcgaaagaccccaagcttcaggtgtgtatatgggtaatttgacac

SEQ ID NO: 15 - DNA, 54 bp, Artificial Sequence
  Description of Artificial Sequence Synthetic oligonucleotide
tcgagtgtcaaattacccatatacacacctgaagcttggggtctttcgctagct

SEQ ID NO: 16 - DNA, 71 bp, Artificial Sequence
  Description of Artificial Sequence Synthetic oligonucleotide
tcgaggctagccagctttgaattttgaccttcttaagcttgcgggagacgtcgagtccaaccctgggccct

SEQ ID NO: 17 - DNA, 72 bp, Artificial Sequence
  Description of Artificial Sequence Synthetic oligonucleotide
tcgaagggcccagggttggactcgacgtctcccgcaagcttaagaaggtcaaaattcaacagctggctagcc

SEQ ID NO: 18 - DNA, 8 bp, Artificial Sequence
  Description of Artificial Sequence Synthetic oligonucleotide
cgagcatg

SEQ ID NO: 19 - DNA, 16 bp, Artificial Sequence
  Description of Artificial Sequence Synthetic oligonucleotide
ctagcatgctcgagct

SEQ ID NO: 20 - DNA, 25 bp, Artificial Sequence
  Description of Artificial Sequence Synthetic oligonucleotide
ctggatccaaaatgaaggcaaacct

SEQ ID NO: 21 - DNA, 28 bp, Artificial Sequence
  Description of Artificial Sequence Synthetic oligonucleotide
caggatcctagatgcatattctgcactg

SEQ ID NO: 22 - DNA, 25 bp, Artificial Sequence
  Description of Artificial Sequence Synthetic oligonucleotide
gaaaggcaaacctactggtcctgtt

SEQ ID NO: 23 - DNA, 37 bp, Artificial Sequence
  Description of Artificial Sequence Synthetic oligonucleotide
cgtgcagtcgacaggatgcatattctgcactgcaaag

SEQ ID NO: 24 - protein, 9 aa, Artificial Sequence
  Description of Artificial Sequence Synthetic peptide
ASNENMETM

SEQ ID NO: 25 - protein, 9 aa, Artificial Sequence
  Description of Artificial Sequence Synthetic peptide
RPQASGVYM

SEQ ID NO: 26 - DNA, 8017 bp, Artificial Sequence
  Description of Artificial Sequence Synthetic plasmid sequence
tttgaaagccgggggtgggagatccggattgccggtccgctcgatatcgcgggccgggtccgtgactacccactccccctttcaacgtgaaggctacgatagtgccagggcgggtcctgccgaaagtgccaacccaaaaccacataaccccccccccccctcccccccccctcacattactggccgaagccgcttggaataaggccggtgtgcgtctgtctatatgttacttctactacattgtcgtctgtgacgatgtaggggcccggaacctggtcctgtcttcttgacgagtattcctaggggtctttcccctctcgacaaaggaatacaaggtctgttgaatgtcgtgaaggaagcagttcctctggacgcttcttgaagacaagcaacgtctgtagcgaccctttgcaggcagcggaatcccccacctggtgacaggtgcctctgcggccgaaagccacgtgtgtaagacacacctgcaaaggcggcacaaccccagtgccacgttgtgcgttggatagttgtggaaagagtcaaatggctctcctcaagcgtattcaacaaggggctgaaggatgcccagaaggtaccccactggctgggatctgatctggggcctcggtgcgcgtgctttacacgcgttgagtcgaggttaaaaaacgtctaggccccccgaaccacggggacgtggttttcctttgaaaaccacgacaataatatggctacaaccatggagctcgagaatacagaggagatggagaatttatcagaccgagtgtctcaagacactgccggcaacacggtcacaaacacccaatcaaccgttggtcgtcttgtcggatacggaacagttcatgatggggaacatccattcgaaacacattatgcaggatacttttcagatcttttgatccacgatgtcgagaccaatcccgggcctttcacgtttaaaccaagacaacggccggtttttcagactcaaggagcggcagtgtcatcaatggctcaaaccctactgccgaacgacttggccagcaaagctatgggatcagcctttacggctttgctcgatgccaacgaggacgcccaaaaagcaatgaagattataaagacgttaagttctctatcggatgcatgggaaaatgtaaaaggaacattgaacaacccggagttctggaaacaactcttaagcagatgtgtgcaactgattgccgggatgacgatagcagtgatgcatccggaccccttgacgctgctttgcttgggagtcttgacagcagcagagatcacaagccagacaagcctgtgcgaagaaatagcagctaaattcaaaacaatcttcactactcccccccctcgttttcctgtgatctcacttttccaacagcagtccccccttaaacaggtcaatgatgttttctctctggcaaagaacctagactgggcagtgaagacagttgaaaaagtggttgattggtttggaacttgggttgcacaagaagagagagagcagaccctggatcagctgctccagcgattccccgagcacgcgaagaggatttcagaccttcgtaatggaatggctgcctatgttgaatgcaaggagagcttcgatttctttgagaaactttacaatcaagcagttaaggagaagagaactggaattgctgccgtttgtgaaaagttcagacaaaaacatgaccatgccacggcacgatgtgaaccagttgtgatcgtgttgcgcggtgatgctggtcagggaaagtcattgtcaagtcaaatcattgcccaggctgtttctaaaactatttttgggcgccagtcagtctattctcttcctcctgattcagatttctttgatggctatgagaaccagtttgccgcaataatggatgatttgggacaaaatcccgatggttcagattttaccaccttctgccagatggtgtccacgacaaacttactcccaaacatggctagtctggagagaaaaggaacccccttcacatctcagctcgtagtggctacgacaaatctcccggagtttagacctgttacaattgcccattatcctgctgttgagcgccgcattactttcgactactcggtgtctgcaggtccagtttgttcaaagaccgaagctggttgcaaagtgttggatgttgaaagagcctttaggccaacaggtgatgcccctcttccatgtttccaaaataattgcctattcttggaaaaggctggcctgcagttcagagataataggtccaaggagattttatctttggttgatgtgatcgagagagctgtgactagaatagagaggaagaagaaagtcctcacagcggtgcagacccttgtggcccaagggcctgttgatgaagttagcttttactcggttgtccagcagctcaaggctagacaggaagctacagatgagcagttggaggaactccaggaagcctttgcccgggttcaggagcggagttcagtgttctcagactggatgaagatttccgccatgctttgtgccgccaccctagctctcacacaagtggtgaagatggctaaggctgtcaaacagatggtgagaccagacttggtgcgggtccagctggatgagcaagaacagggtccttataacgaaaccacccgtataaagcccaaaactcttcaattgctagatgtccagggtccaaatccgactatggactttgaaaagtttgttgctaagtttgttacagcccccattggttttgtgtaccccacaggtgttagcactcagacatgcctacttgtgaagggacgtaccctggcggtgaatcggcacatggcagagtctgactggacctccattgtagtgcgtggtgttagccacacccgctcctcagtgaaaattatcgccatagccaaagctgggaaggagactgatgtgtcgttcattcgcctttcatctggtcccttgtttagagataatactagcaagtttgtgaaggccagtgacgtattgccccatagctcttccccccttattgggatcatgaatgtggacattccaatgatgtatacagggacatttctgaaggctggcgtctcggtgccggttgagacagggcagactttcaaccactgcatccactacaaagcaaatacacggaaaggctggtgtgggtctgcaatcctggccgatcttggtgggagcaagaagattctgggcttccattcagccggctccatgggcgttgcagccgcgtcgataatttcacaagaaatgatcgatgcggtggtgcaggccttcgagccccagggtgcacttgagcggctgccagatggtccgcgcatccatgtaccccgaaagactgctttgcgcccgactgttgccagacaggtcttccaacccgcttttgccccagctgttctttctaaatttgacccacgcacggatgctgatgttgacgaagtagctttttcaaaacatacatccaatcaggaaaccctccccccagtgtttagaatggttgctagggaatatgcgaacagagtattcgcactgttgggcagagacaatggaaggctgtcagtcaagcaagccttggatggacttgaggggatggaccctatggacaagaacacttccccaggccttccatatactacgctaggaatgcgtagaacagatgttgtagattgggaaaccgccactcttatcccctttgcagcagagagactagaaaaaatgaataacaaagacttttccgacattgtctatcagacattcctcaaggacgagcttagacctatagagaaggtacaagccgccaagacacggattgtggatgttccaccatttgagcactgcattctgggtagacaactgctcgggaagttcgcttccaaattccagacccaaccgggtctggaattgggctctgcaattgggtgtgacccagacgtgcattggacagcctttggtgtggcaatgcaaggctttgaaagggtgtatgatgtggattattccaattttgattctacccattcagtagctatatttaggttattggcagaggaattcttttctgaagagaatggcttcgacccattggttaaggattatcttgagtccttagccatttcaaaacatgcgtatgaggaaaagcgctatctcataaccggtggtcttccgtctggttgtgcagcgacctcaatgttaaatacaataatgaataatattattattagggccggtttgtatcttacatataaaaattttgagtttgatgacgtgaaggtcttgtcttatggtgatgatcttctagtggcaactaattaccaattgaactttgatagagtgagaacaagcctggcaaagacaggatataagattacacccgctaacaaaacttctacctttcccctggaatcaactcttgaggatgtagtattcctgaagagaaaatttaagaaagagggccctctatatcgacctgtcatgaatagagaggcgttagaagcaatgttgtcatattatcgtccagggactctatctgagaaactcacttcaatcactatgcttgccgtgcattctggcaaacaggagtacgatcgactctttgccccgtttcgcgaggttggagtgatcgtaccaacttttgagagtgtggagtacagatggaggagcctgttctggtaatagcgcggtcactggcacaacgcgttacccggtaagccaaccgggtgtacacggtcgtcataccgcagacagggttcttctactttgcaagataaactagagtagtaaaataaatagttttaaaaaaaaaaaaaaaaaaaaaaacgggatcctctagagtcgacctgcaggcatgcaagcttttgttccctttagtgagggttaattccgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagg

SEQ ID NO: 27 - DNA, 8092 bp, Artificial Sequence
  Description of Artificial Sequence Synthetic plasmid sequence
tttgaaagccgggggtgggagatccggattgccggtccgctcgatatcgcgggccgggtccgtgactacccactccccctttcaacgtgaaggctacgatagtgccagggcgggtcctgccgaaagtgccaacccaaaaccacataaccccccccccccctcccccccccctcacattactggccgaagccgcttggaataaggccggtgtgcgtctgtctatatgttacttctactacattgtcgtctgtgacgatgtaggggcccggaacctggtcctgtcttcttgacgagtattcctaggggtctttcccctctcgacaaaggaatacaaggtctgttgaatgtcgtgaaggaagcagttcctctggacgcttcttgaagacaagcaacgtctgtagcgaccctttgcaggcagcggaatcccccacctggtgacaggtgcctctgcggccgaaagccacgtgtgtaagacacacctgcaaaggcggcacaaccccagtgccacgttgtgcgttggatagttgtggaaagagtcaaatggctctcctcaagcgtattcaacaaggggctgaaggatgcccagaaggtaccccactggctgggatctgatctggggcctcggtgcgcgtgctttacacgcgttgagtcgaggttaaaaaacgtctaggccccccgaaccacggggacgtggttttcctttgaaaaccacgacaataatatggctacaaccatggagctcgagcatgctagccagctgttgaattttgaccttcttaagcttgcgggagacgtcgagtccaaccctgggcccttcgagaatacagaggagatggagaatttatcagaccgagtgtctcaagacactgccggcaacacggtcacaaacacccaatcaaccgttggtcgtcttgtcggatacggaacagttcatgatggggaacatccattcgaaacacattatgcaggatacttttcagatcttttgatccacgatgtcgagaccaatcccgggcctttcacgtttaaaccaagacaacggccggtttttcagactcaaggagcggcagtgtcatcaatggctcaaaccctactgccgaacgacttggccagcaaagctatgggatcagcctttacggctttgctcgatgccaacgaggacgcccaaaaagcaatgaagattataaagacgttaagttctctatcggatgcatgggaaaatgtaaaaggaacattgaacaacccggagttctggaaacaactcttaagcagatgtgtgcaactgattgccgggatgacgatagcagtgatgcatccggaccccttgacgctgctttgcttgggagtcttgacagcagcagagatcacaagccagacaagcctgtgcgaagaaatagcagctaaattcaaaacaatcttcactactcccccccctcgttttcctgtgatctcacttttccaacagcagtccccccttaaacaggtcaatgatgttttctctctggcaaagaacctagactgggcagtgaagacagttgaaaaagtggttgattggtttggaacttgggttgcacaagaagagagagagcagaccctggatcagctgctccagcgattccccgagcacgcgaagaggatttcagaccttcgtaatggaatggctgcctatgttgaatgcaaggagagcttcgatttctttgagaaactttacaatcaagcagttaaggagaagagaactggaattgctgccgtttgtgaaaagttcagacaaaaacatgaccatgccacggcacgatgtgaaccagttgtgatcgtgttgcgcggtgatgctggtcagggaaagtcattgtcaagtcaaatcattgcccaggctgtttctaaaactatttttgggcgccagtcagtctattctcttcctcctgattcagatttctttgatggctatgagaaccagtttgccgcaataatggatgatttgggacaaaatcccgatggttcagattttaccaccttctgccagatggtgtccacgacaaacttactcccaaacatggctagtctggagagaaaaggaacccccttcacatctcagctcgtagtggctacgacaaatctcccggagtttagacctgttacaattgcccattatcctgctgttgagcgccgcattactttcgactactcggtgtctgcaggtccagtttgttcaaagaccgaagctggttgcaaagtgttggatgttgaaagagcctttaggccaacaggtgatgcccctcttccatgtttccaaaataattgcctattcttggaaaaggctggcctgcagttcagagataataggtccaaggagattttatctttggttgatgtgatcgagagagctgtgactagaatagagaggaagaagaaagtcctcacagcggtgcagacccttgtggcccaagggcctgttgatgaagttagcttttactcggttgtccagcagctcaaggctagacaggaagctacagatgagcagttggaggaactccaggaagcctttgcccgggttcaggagcggagttcagtgttctcagactggatgaagatttccgccatgctttgtgccgccaccctagctctcacacaagtggtgaagatggctaaggctgtcaaacagatggtgagaccagacttggtgcgggtccagctggatgagcaagaacagggtccttataacgaaaccacccgtataaagcccaaaactcttcaattgctagatgtccagggtccaaatccgactatggactttgaaaagtttgttgctaagtttgttacagcccccattggttttgtgtaccccacaggtgttagcactcagacatgcctacttgtgaagggacgtaccctggcggtgaatcggcacatggcagagtctgactggacctccattgtagtgcgtggtgttagccacacccgctcctcagtgaaaattatcgccatagccaaagctgggaaggagactgatgtgtcgttcattcgcctttcatctggtcccttgtttagagataatactagcaagtttgtgaaggccagtgacgtattgccccatagctcttccccccttattgggatcatgaatgtggacattccaatgatgtatacagggacatttctgaaggctggcgtctcggtgccggttgagacagggcagactttcaaccactgcatccactacaaagcaaatacacggaaaggctggtgtgggtctgcaatcctggccgatcttggtgggagcaagaagattctgggcttccattcagccggctccatgggcgttgcagccgcgtcgataatttcacaagaaatgatcgatgcggtggtgcaggccttcgagccccagggtgcacttgagcggctgccagatggtccgcgcatccatgtaccccgaaagactgctttgcgcccgactgttgccagacaggtcttccaacccgcttttgccccagctgttctttctaaatttgacccacgcacggatgctgatgttgacgaagtagctttttcaaaacatacatccaatcaggaaaccctccccccagtgtttagaatggttgctagggaatatgcgaacagagtattcgcactgttgggcagagacaatggaaggctgtcagtcaagcaagccttggatggacttgaggggatggaccctatggacaagaacacttccccaggccttccatatactacgctaggaatgcgtagaacagatgttgtagattgggaaaccgccactcttatcccctttgcagcagagagactagaaaaaatgaataacaaagacttttccgacattgtctatcagacattcctcaaggacgagcttagacctatagagaaggtacaagccgccaagacacggattgtggatgttccaccatttgagcactgcattctgggtagacaactgctcgggaagttcgcttccaaattccagacccaaccgggtctggaattgggctctgcaattgggtgtgacccagacgtgcattggacagcctttggtgtggcaatgcaaggctttgaaagggtgtatgatgtggattattccaattttgattctacccattcagtagctatatttaggttattggcagaggaattcttttctgaagagaatggcttcgacccattggttaaggattatcttgagtccttagccatttcaaaacatgcgtatgaggaaaagcgctatctcataaccggtggtcttccgtctggttgtgcagcgacctcaatgttaaatacaataatgaataatattattattagggccggtttgtatcttacatataaaaattttgagtttgatgacgtgaaggtcttgtcttatggtgatgatcttctagtggcaactaattaccaattgaactttgatagagtgagaacaagcctggcaaagacaggatataagattacacccgctaacaaaacttctacctttcccctggaatcaactcttgaggatgtagtattcctgaagagaaaatttaagaaagagggccctctatatcgacctgtcatgaatagagaggcgttagaagcaatgttgtcatattatcgtccagggactctatctgagaaactcacttcaatcactatgcttgccgtgcattctggcaaacaggagtacgatcgactctttgccccgtttcgcgaggttggagtgatcgtaccaacttttgagagtgtggagtacagatggaggagcctgttctggtaatagcgcggtcactggcacaacgcgttacccggtaagccaaccgggtgtacacggtcgtcataccgcagacagggttcttctactttgcaagataaactagagtagtaaaataaatagttttaaaaaaaaaaaaaaaaaaaaaaacgggatcctctagagtcgacctgcaggcatgcaagcttttgttccctttagtgagggttaattccgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagg

SEQ ID NO: 28 - DNA, 10417 bp, Artificial Sequence
  Description of Artificial Sequence Synthetic plasmid sequence
tttgaaagccgggggtgggagatccggattgccggtccgctcgatatcgcgggccgggtccgtgactacccactccccctttcaacgtgaaggctacgatagtgccagggcgggtcctgccgaaagtgccaacccaaaaccacataaccccccccccccctcccccccccctcacattactggccgaagccgcttggaataaggccggtgtgcgtctgtctatatgttacttctactacattgtcgtctgtgacgatgtaggggcccggaacctggtcctgtcttcttgacgagtattcctaggggtctttcccctctcgacaaaggaatacaaggtctgttgaatgtcgtgaaggaagcagttcctctggacgcttcttgaagacaagcaacgtctgtagcgaccctttgcaggcagcggaatcccccacctggtgacaggtgcctctgcggccgaaagccacgtgtgtaagacacacctgcaaaggcggcacaaccccagtgccacgttgtgcgttggatagttgtggaaagagtcaaatggctctcctcaagcgtattcaacaaggggctgaaggatgcccagaaggtaccccactggctgggatctgatctggggcctcggtgcgcgtgctttacacgcgttgagtcgaggttaaaaaacgtctaggccccccgaaccacggggacgtggttttcctttgaaaaccacgacaataatatggctacaaccatggagctcatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaaggagctcgagatgtccttgtctaaggaagttaagagcttccaatggacgcaagcattgagaagagaattgcagagcttcacatcagatgtgaaggctgctgtcattaaggatgcaaccaaccttctgaatgggttggacttctctgaggtcagcaatgttcagaggatcatgaggaaggaaaagagagatgacaaagacctacagagactcagaagtctcaaccagactgtacattctcttgtggatttaaagtcaacatcaaagaagaatgttttgaaagtggggaggctcagtgcagaagaactgatgtctcttgcggctgaccttgagaagctgaaggccaagatcatgaggtctgaaaggccccaggcttcaggggtatatatggggaacttaacaacacagcaactagaccaaagatctcagatcctacagatagttgggatgagaaagcctcagcagggtgcaagtggtgtggtaagagtttgggatgtgaaagactcatcacttttgaacaatcaatttggcacaatgccaagtctaactatggcttgtatggccaaacagtcacagactccgctcaatgacgttgtacaagcgctcacagaccttggcttgctttacacagtcaagtatccaaatcttaatgatcttgaaaggctgaaagacaagcacccagttctgggggtcatcactgaacagcagtccagcatcaacatttctggctataactttagtcttggtgctgccgtgaaggcaggggcagccctgttggatgggggtaacatgttagagtcaattttgatcaagccaagcaacagcgaggacctcttgaaggcagttctcggggccaagagaaaactcaacatgtttgtttcagaccaagttggggacaggaacccttatgaaaacatcctctataaagtttgcctttcaggtgaaggatggccatacatagcttgtagaacatcgattgtggggagagcatgggaaaacacaacaattgatctcacaagcgagaaacctgcagtcaactcacccaggccagcgcctggagcagcaggtccacctcaggtgggcttaagctacagccagacaatgcttttaaaagacctcatgggaggaattgaccccaacgctcctacatggattgacattgagggtagatttaatgatccagtggaaatagcaattttccaaccacagaacgggcagttcatacacttttacagggaacccgttgatcaaaaacaattcaagcaagattccaagtactcacacggcatggatcttgccgacctcttcaatgcgcaacccgggttgacctcgtcagttataggtgctcttccgcaggggatggttctaagctgtcaaggctccgatgacatcagaaagcttctggactcacaaaataggaaggacattaagcttatcgatgttgaaatgaccagggaagcttcgagggagtatgaagacaaagtgtgggacaaatatggctggttgtgtaagatgcatactggaatagtaagggacaaaaagaagaaagagatcaccccgcactgtgcactcatggactgcatcatttttgaaagcgcctccaaagcaaggctcccagatctgaaaactgttcacaacattctgccacatgacctaatttttagaggcccaaatgttgtgacactcgagaatacagaggagatggagaatttatcagaccgagtgtctcaagacactgccggcaacacggtcacaaacacccaatcaaccgttggtcgtcttgtcggatacggaacagttcatgatggggaacatccattcgaaacacattatgcaggatacttttcagatcttttgatccacgatgtcgagaccaatcccgggcctttcacgtttaaaccaagacaacggccggtttttcagactcaaggagcggcagtgtcatcaatggctcaaaccctactgccgaacgacttggccagcaaagctatgggatcagcctttacggctttgctcgatgccaacgaggacgcccaaaaagcaatgaagattataaagacgttaagttctctatcggatgcatgggaaaatgtaaaaggaacattgaacaacccggagttctggaaacaactcttaagcagatgtgtgcaactgattgccgggatgacgatagcagtgatgcatccggaccccttgacgctgctttgcttgggagtcttgacagcagcagagatcacaagccagacaagcctgtgcgaagaaatagcagctaaattcaaaacaatcttcactactcccccccctcgttttcctgtgatctcacttttccaacagcagtccccccttaaacaggtcaatgatgttttctctctggcaaagaacctagactgggcagtgaagacagttgaaaaagtggttgattggtttggaacttgggttgcacaagaagagagagagcagaccctggatcagctgctccagcgattccccgagcacgcgaagaggatttcagaccttcgtaatggaatggctgcctatgttgaatgcaaggagagcttcgatttctttgagaaactttacaatcaagcagttaaggagaagagaactggaattgctgccgtttgtgaaaagttcagacaaaaacatgaccatgccacggcacgatgtgaaccagttgtgatcgtgttgcgcggtgatgctggtcagggaaagtcattgtcaagtcaaatcattgcccaggctgtttctaaaactatttttgggcgccagtcagtctattctcttcctcctgattcagatttctttgatggctatgagaaccagtttgccgcaataatggatgatttgggacaaaatcccgatggttcagattttaccaccttctgccagatggtgtccacgacaaacttactcccaaacatggctagtctggagagaaaaggaacccccttcacatctcagctcgtagtggctacgacaaatctcccggagtttagacctgttacaattgcccattatcctgctgttgagcgccgcattactttcgactactcggtgtctgcaggtccagtttgttcaaagaccgaagctggttgcaaagtgttggatgttgaaagagcctttaggccaacaggtgatgcccctcttccatgtttccaaaataattgcctattcttggaaaaggctggcctgcagttcagagataataggtccaaggagattttatctttggttgatgtgatcgagagagctgtgactagaatagagaggaagaagaaagtcctcacagcggtgcagacccttgtggcccaagggcctgttgatgaagttagcttttactcggttgtccagcagctcaaggctagacaggaagctacagatgagcagttggaggaactccaggaagcctttgcccgggttcaggagcggagttcagtgttctcagactggatgaagatttccgccatgctttgtgccgccaccctagctctcacacaagtggtgaagatggctaaggctgtcaaacagatggtgagaccagacttggtgcgggtccagctggatgagcaagaacagggtccttataacgaaaccacccgtataaagcccaaaactcttcaattgctagatgtccagggtccaaatccgactatggactttgaaaagtttgttgctaagtttgttacagcccccattggttttgtgtaccccacaggtgttagcactcagacatgcctacttgtgaagggacgtaccctggcggtgaatcggcacatggcagagtctgactggacctccattgtagtgcgtggtgttagccacacccgctcctcagtgaaaattatcgccatagccaaagctgggaaggagactgatgtgtcgttcattcgcctttcatctggtcccttgtttagagataatactagcaagtttgtgaaggccagtgacgtattgccccatagctcttccccccttattgggatcatgaatgtggacattccaatgatgtatacagggacatttctgaaggctggcgtctcggtgccggttgagacagggcagactttcaaccactgcatccactacaaagcaaatacacggaaaggctggtgtgggtctgcaatcctggccgatcttggtgggagcaagaagattctgggcttccattcagccggctccatgggcgttgcagccgcgtcgataatttcacaagaaatgatcgatgcggtggtgcaggccttcgagccccagggtgcacttgagcggctgccagatggtccgcgcatccatgtaccccgaaagactgctttgcgcccgactgttgccagacaggtcttccaacccgcttttgccccagctgttctttctaaatttgacccacgcacggatgctgatgttgacgaagtagctttttcaaaacatacatccaatcaggaaaccctccccccagtgtttagaatggttgctagggaatatgcgaacagagtattcgcactgttgggcagagacaatggaaggctgtcagtcaagcaagccttggatggacttgaggggatggaccctatggacaagaacacttccccaggccttccatatactacgctaggaatgcgtagaacagatgttgtagattgggaaaccgccactcttatcccctttgcagcagagagactagaaaaaatgaataacaaagacttttccgacattgtctatcagacattcctcaaggacgagcttagacctatagagaaggtacaagccgccaagacacggattgtggatgttccaccatttgagcactgcattctgggtagacaactgctcgggaagttcgcttccaaattccagacccaaccgggtctggaattgggctctgcaattgggtgtgacccagacgtgcattggacagcctttggtgtggcaatgcaaggctttgaaagggtgtatgatgtggattattccaattttgattctacccattcagtagctatatttaggttattggcagaggaattcttttctgaagagaatggcttcgacccattggttaaggattatcttgagtccttagccatttcaaaacatgcgtatgaggaaaagcgctatctcataaccggtggtcttccgtctggttgtgcagcgacctcaatgttaaatacaataatgaataatattattattagggccggtttgtatcttacatataaaaattttgagtttgatgacgtgaaggtcttgtcttatggtgatgatcttctagtggcaactaattaccaattgaactttgatagagtgagaacaagcctggcaaagacaggatataagattacacccgctaacaaaacttctacctttcccctggaatcaactcttgaggatgtagtattcctgaagagaaaatttaagaaagagggccctctatatcgacctgtcatgaatagagaggcgttagaagcaatgttgtcatattatcgtccagggactctatctgagaaactcacttcaatcactatgcttgccgtgcattctggcaaacaggagtacgatcgactctttgccccgtttcgcgaggttggagtgatcgtaccaacttttgagagtgtggagtacagatggaggagcctgttctggtaatagcgcggtcactggcacaacgcgttacccggtaagccaaccgggtgtacacggtcgtcataccgcagacagggttcttctactttgcaagataaactagagtagtaaaataaatagttttaaaaaaaaaaaaaaaaaaaaaaacgggatcctctagagtcgacctgcaggcatgcaagcttttgttccctttagtgagggttaattccgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagg